SEQUENCE LISTING (33 sequences)

SEQ ID NO: 1 - DNA, 1365 bp, Homo sapiens
  misc_feature: TNFRSF1A
atgggcctctccaccgtgcctgacctgctgctgccgctggtgctcctggagctgttggtgggaatatacccctcaggggttattggactggtccctcacctaggggacagggagaagagagatagtgtgtgtccccaaggaaaatatatccaccctcaaaataattcgatttgctgtaccaagtgccacaaaggaacctacttgtacaatgactgtccaggcccggggcaggatacggactgcagggagtgtgagagcggctccttcaccgcttcagaaaaccacctcagacactgcctcagctgctccaaatgccgaaaggaaatgggtcaggtggagatctcttcttgcacagtggaccgggacaccgtgtgtggctgcaggaagaaccagtaccggcattattggagtgaaaaccttttccagtgcttcaattgcagcctctgcctcaatgggaccgtgcacctctcctgccaggagaaacagaacaccgtgtgcacctgccatgcaggtttctttctaagagaaaacgagtgtgtctcctgtagtaactgtaagaaaagcctggagtgcacgaagttgtgcctaccccagattgagaatgttaagggcactgaggactcaggcaccacagtgctgttgcccctggtcattttctttggtctttgccttttatccctcctcttcattggtttaatgtatcgctaccaacggtggaagtccaagctctactccattgtttgtgggaaatcgacacctgaaaaagagggggagcttgaaggaactactactaagcccctggccccaaacccaagcttcagtcccactccaggcttcacccccaccctgggcttcagtcccgtgcccagttccaccttcacctccagctccacctatacccccggtgactgtcccaactttgcggctccccgcagagaggtggcaccaccctatcagggggctgaccccatccttgcgacagccctcgcctccgaccccatccccaacccccttcagaagtgggaggacagcgcccacaagccacagagcctagacactgatgaccccgcgacgctgtacgccgtggtggagaacgtgcccccgttgcgctggaaggaattcgtgcggcgcctagggctgagcgaccacgagatcgatcggctggagctgcagaacgggcgctgcctgcgcgaggcgcaatacagcatgctggcgacctggaggcggcgcacgccgcggcgcgaggccacgctggagctgctgggacgcgtgctccgcgacatggacctgctgggctgcctggaggacatcgaggaggcgctttgcggccccgccgccctcccgcccgcgcccagtcttctcaga

SEQ ID NO: 2 - protein, 455 aa, Homo sapiens
  MISC_FEATURE: Wild Type TNF Receptor 1
MGLSTVPDLLLPLVLLELLVGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTTVLLPLVIFFGLCLLSLLFIGLMYRYQRWKSKLYSIVCGKSTPEKEGELEGTTTKPLAPNPSFSPTPGFTPTLGFSPVPSSTFTSSSTYTPGDCPNFAAPRREVAPPYQGADPILATALASDPIPNPLQKWEDSAHKPQSLDTDDPATLYAVVENVPPLRWKEFVRRLGLSDHEIDRLELQNGRCLREAQYSMLATWRRRTPRREATLELLGRVLRDMDLLGCLEDIEEALCGPAALPPAPSLLR

SEQ ID NO: 3 - DNA, 591 bp, Homo sapiens
  misc_feature: Ligand Binding Domain of TNFR1
atgggcctctccaccgtgcctgacctgctgctgccgctggtgctcctggagctgttggtgggaatatacccctcaggggttattggactggtccctcacctaggggacagggagaagagagatagtgtgtgtccccaaggaaaatatatccaccctcaaaataattcgatttgctgtaccaagtgccacaaaggaacctacttgtacaatgactgtccaggcccggggcaggatacggactgcagggagtgtgagagcggctccttcaccgcttcagaaaaccacctcagacactgcctcagctgctccaaatgccgaaaggaaatgggtcaggtggagatctcttcttgcacagtggaccgggacaccgtgtgtggctgcaggaagaaccagtaccggcattattggagtgaaaaccttttccagtgcttcaattgcagcctctgcctcaatgggaccgtgcacctctcctgccaggagaaacagaacaccgtgtgcacctgccatgcaggtttctttctaagagaaaacgagtgtgtctcctgtagtaactgtaagaaaagcctggagtgcacgaagttgtgcctacca

SEQ ID NO: 4 - protein, 197 aa, Homo sapiens
  MISC_FEATURE: Ligand Binding Domain of TNFR1
MGLSTVPDLLLPLVLLELLVGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLP

SEQ ID NO: 5 - DNA, 1008 bp, Homo sapiens
  misc_feature: full-length FAS
atgctgggcatctggaccctcctacctctggttcttacgtctgttgctagattatcgtccaaaagtgttaatgcccaagtgactgacatcaactccaagggattggaattgaggaagactgttactacagttgagactcagaacttggaaggcctgcatcatgatggccaattctgccataagccctgtcctccaggtgaaaggaaagctagggactgcacagtcaatggggatgaaccagactgcgtgccctgccaagaagggaaggagtacacagacaaagcccatttttcttccaaatgcagaagatgtagattgtgtgatgaaggacatggcttagaagtggaaataaactgcacccggacccagaataccaagtgcagatgtaaaccaaactttttttgtaactctactgtatgtgaacactgtgacccttgcaccaaatgtgaacatggaatcatcaaggaatgcacactcaccagcaacaccaagtgcaaagaggaaggatccagatctaacttggggtggctttgtcttcttcttttgccaattccactaattgtttgggtgaagagaaaggaagtacagaaaacatgcagaaagcacagaaaggaaaaccaaggttctcatgaatctccaactttaaatcctgaaacagtggcaataaatttatctgatgttgacttgagtaaatatatcaccactattgctggagtcatgacactaagtcaagttaaaggctttgttcgaaagaatggtgtcaatgaagccaaaatagatgagatcaagaatgacaatgtccaagacacagcagaacagaaagttcaactgcttcgtaattggcatcaacttcatggaaagaaagaagcgtatgacacattgattaaagatctcaaaaaagccaatctttgtactcttgcagagaaaattcagactatcatcctcaaggacattactagtgactcagaaaattcaaacttcagaaatgaaatccaaagcttggtctag

SEQ ID NO: 6 - protein, 335 aa, Homo sapiens
  MISC_FEATURE: full-length FAS
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVKRKEVQKTCRKHRKENQGSHESPTLNPETVAINLSDVDLSKYITTIAGVMTLSQVKGFVRKNGVNEAKIDEIKNDNVQDTAEQKVQLLRNWHQLHGKKEAYDTLIKDLKKANLCTLAEKIQTIILKDITSDSENSNFRNEIQSLV

SEQ ID NO: 7 - DNA, 505 bp, Homo sapiens
  misc_feature: Effector Domain of FAS
aggatccagatctaacttggggtggctttgtcttcttcttttgccaattccactaattgtttgggtgaagagaaaggaagtacagaaaacatgcagaaagcacagaaaggaaaaccaaggttctcatgaatctccaaccttaaatcctgaaacagtggcaataaatttatctgatgttgacttgagtaaatatatcaccactattgctggagtcatgacactaagtcaagttaaaggctttgttcgaaagaatggtgtcaatgaagccaaaatagatgagatcaagaatgacaatgtccaagacacagcagaacagaaagttcaactgcttcgtaattggcatcaacttcatggaaagaaagaagcgtatgacacattgattaaagatctcaaaaaagccaatctttgtactcttgcagagaaaattcagactatcatcctcaaggacattactagtgactcagaaaattcaaacttcagaaatgaaatccaaagcttggtctag

SEQ ID NO: 8 - protein, 167 aa, Homo sapiens
  MISC_FEATURE: Effector Domain of FAS
GSRSNLGWLCLLLLPIPLIVWVKRKEVQKTCRKHRKENQGSHESPTLNPETVAINLSDVDLSKYITTIAGVMTLSQVKGFVRKNGVNEAKIDEIKNDNVQDTAEQKVQLLRNWHQLHGKKEAYDTLIKDLKKANLCTLAEKIQTIILKDITSDSENSNFRNEIQSLV

SEQ ID NO: 9 - DNA, 1101 bp, Homo sapiens
  misc_feature: FAS-chimera
atgggcctctccaccgtgcctgacctgctgctgccgctggtgctcctggagctgttggtgggaatatacccctcaggggttattggactggtccctcacctaggggacagggagaagagagatagtgtgtgtccccaaggaaaatatatccaccctcaaaataattcgatttgctgtaccaagtgccacaaaggaacctacttgtacaatgactgtccaggcccggggcaggatacggactgcagggagtgtgagagcggctccttcaccgcttcagaaaaccacctcagacactgcctcagctgctccaaatgccgaaaggaaatgggtcaggtggagatctcttcttgcacagtggaccgggacaccgtgtgtggctgcaggaagaaccagtaccggcattattggagtgaaaaccttttccagtgcttcaattgcagcctctgcctcaatgggaccgtgcacctctcctgccaggagaaacagaacaccgtgtgcacctgccatgcaggtttctttctaagagaaaacgagtgtgtctcctgtagtaactgtaagaaaagcctggagtgcacgaagttgtgcctaccaagcttaggatccagatctaacttggggtggctttgtcttcttcttttgccaattccactaattgtttgggtgaagagaaaggaagtacagaaaacatgcagaaagcacagaaaggaaaaccaaggttctcatgaatctccaaccttaaatcctgaaacagtggcaataaatttatctgatgttgacttgagtaaatatatcaccactattgctggagtcatgacactaagtcaagttaaaggctttgttcgaaagaatggtgtcaatgaagccaaaatagatgagatcaagaatgacaatgtccaagacacagcagaacagaaagttcaactgcttcgtaattggcatcaacttcatggaaagaaagaagcgtatgacacattgattaaagatctcaaaaaagccaatctttgtactcttgcagagaaaattcagactatcatcctcaaggacattactagtgactcagaaaattcaaacttcagaaatgaaatccaaagcttggtctag

SEQ ID NO: 10 - protein, 366 aa, Homo sapiens
  MISC_FEATURE: FAS-chimera
MGLSTVPDLLLPLVLLELLVGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPSLGSRSNLGWLCLLLLPIPLIVWVKRKEVQKTCRKHRKENQGSHESPTLNPETVAINLSDVDLSKYITTIAGVMTLSQVKGFVRKNGVNEAKIDEIKNDNVQDTAEQKVQLLRNWHQLHGKKEAYDTLIKDLKKANLCTLAEKIQTIILKDITSDSENSNFRNEIQSLV

SEQ ID NO: 11 - DNA, 47 bp, Artificial Sequence
  Endothelial Cell-Specific Enhancer Elements
ctggagggtgactttgcttctggagccagtacttcatacttttcatt

SEQ ID NO: 12 - DNA, 47 bp, Artificial Sequence
  Endothelial Cell-Specific Enhancer
aatgaaaagtatgaagtactggctccagaagcaaagtcaccctccag

SEQ ID NO: 13 - DNA, 44 bp, Artificial Sequence
  Endothelial Cell-Specific Enhancer
gtacttcatacttttcattccaatggggtgactttgcttctgga

SEQ ID NO: 14 - DNA, 44 bp, Artificial Sequence
  Endothelial Cell-Specific Enhancer Element
tccagaagcaaagtcaccccattggaatgaaaagtatgaagtac

SEQ ID NO: 15 - DNA, 131 bp, Artificial Sequence
  Endothelial Cell-Specific 3X Enhancer Element
ctccagaagcaaagtcaccccattggaatgaaaagtatgaagtacaatgaaaagtatgaagtactggctccagaagcaaagtcaccctccagaagcaaagtcaccccattggaatgaaaagtatgaagtac

SEQ ID NO: 16 - DNA, 131 bp, Artificial Sequence
  Endothelial Cell-Specific 3X Enhancer Element
gtacttcatacttttcattccaatggggtgactttgcttctggagggtgactttgcttctggagccagtacttcatacttttcattgtacttcatacttttcattccaatggggtgactttgcttctggag

SEQ ID NO: 17 - DNA, 850 bp, Artificial Sequence
  Endothelial Cell-Specific PPE-1 Promoter
gtacgtgtacttctgatcggcgatactagggagataaggatgtgcctgacaaaaccacattgttgttgttatcattattatttagttttccttccttgctaactcctgacggaatctttctcacctcaaatgcgaagtactttagtttagaaaagacttggtggaaggggtggtggtggaaaagtagggtgatcttccaaactaatctggttccccgcccgccccagtagctgggattcaagagcgaagagtggggatcgtccccttgtttgatcagaaagacataaaaggaaaatcaagtgaacaatgatcagccccacctccaccccacccccctgcgcgcgcacaatacaatctatttaattgtacttcatacttttcattccaatggggtgactttgcttctggagaaactcttgattcttgaactctggggctggcagctagcaaaaggggaagcgggctgctgctctctgcaggttctgcagcggtctctgtctagtgggtgttttctttttcttagccctgcccctggattgtcagacggcgggcgtctgcctctgaagttagccgtgatttcctctagagccgggtcttatctctggctgcacgttgcctgtgggtgactaatcacacaataacattgtttagggctggaatgaagtcagagctgtttacccccactctataggggttcaatataaaaaggcggcggagaactgtccgagtcagaagcgttcctgcaccggcgctgagagcctgacccggtctgctccgctgtccttgcgcgctgcctcccggctgcccgcgacgctttcgccccagtggaagggccacttgctgcggccgc

SEQ ID NO: 18 - DNA, 987 bp, Artificial Sequence
  Endothelial Cell-Specific PPE-1 3X Promoter
gtacgtgtacttctgatcggcgatactagggagataaggatgtgcctgacaaaaccacattgttgttgttatcattattatttagttttccttccttgctaactcctgacggaatctttctcacctcaaatgcgaagtactttagtttagaaaagacttggtggaaggggtggtggtggaaaagtagggtgatcttccaaactaatctggttccccgcccgccccagtagctgggattcaagagcgaagagtggggatcgtccccttgtttgatcagaaagacataaaaggaaaatcaagtgaacaatgatcagccccacctccaccccacccccctgcgcgcgcacaatacaatctatttaattgtacttcatacttttcattccaatggggtgactttgcttctggagaaactcttgattcttgaactctggggctggcagctagcctccagaagcaaagtcaccccattggaatgaaaagtatgaagtacaatgaaaagtatgaagtactggctccagaagcaaagtcaccctccagaagcaaagtcaccccattggaatgaaaagtatgaagtacgctagcaaaaggggaagcgggctgctgctctctgcaggttctgcagcggtctctgtctagtgggtgttttctttttcttagccctgcccctggattgtcagacggcgggcgtctgcctctgaagttagccgtgatttcctctagagccgggtcttatctctggctgcacgttgcctgtgggtgactaatcacacaataacattgtttagggctggaatgaagtcagagctgtttacccccactctataggggttcaatataaaaaggcggcggagaactgtccgagtcagaagcgttcctgcaccggcgctgagagcctgacccggtctgctccgctgtccttgcgcgctgcctcccggctgcccgcgacgctttcgccccagtggaagggccacttgctgcggccgc

SEQ ID NO: 19 - DNA, 35207 bp, Artificial Sequence
  VB-111 entire construct
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagtacgtacgtgtacttctgatcggcgatactagggagataaggatgtgcctgacaaaaccacattgttgttgttatcattattatttagttttccttccttgctaactcctgacggaatctttctcacctcaaatgcgaagtactttagtttagaaaagacttggtggaaggggtggtggtggaaaagtagggtgatcttccaaactaatctggttccccgcccgccccagtagctgggattcaagagcgaagagtggggatcgtccccttgtttgatcagaaagacataaaaggaaaatcaagtgaacaatgatcagccccacctccaccccacccccctgcgcgcgcacaatacaatctatttaattgtacttcatacttttcattccaatggggtgactttgcttctggagaaactcttgattcttgaactctggggctggcagctagcctccagaagcaaagtcaccccattggaatgaaaagtatgaagtacaatgaaaagtatgaagtactggctccagaagcaaagtcaccctccagaagcaaagtcaccccattggaatgaaaagtatgaagtacgctagcaaaaggggaagcgggctgctgctctctgcaggttctgcagcggtctctgtctagtgggtgttttctttttcttagccctgcccctggattgtcagacggcgggcgtctgcctctgaagttagccgtgatttcctctagagccgggtcttatctctggctgcacgttgcctgtgggtgactaatcacacaataacattgtttagggctggaatgaagtcagagctgtttacccccactctataggggttcaatataaaaaggcggcggagaactgtccgagtcagaagcgttcctgcaccggcgctgagagcctgacccggtctgctccgctgtccttgcgcgctgcctcccggctgcccgcgacgctttcgccccagtggaagggccacttgctgcggccgctaattctgcagatcgggatccggcatgggcctctccaccgtgcctgacctgctgctgccgctggtgctcctggagctgttggtgggaatatacccctcaggggttattggactggtccctcacctaggggacagggagaagagagatagtgtgtgtccccaaggaaaatatatccaccctcaaaataattcgatttgctgtaccaagtgccacaaaggaacctacttgtacaatgactgtccaggcccggggcaggatacggactgcagggagtgtgagagcggctccttcaccgcttcagaaaaccacctcagacactgcctcagctgctccaaatgccgaaaggaaatgggtcaggtggagatctcttcttgcacagtggaccgggacaccgtgtgtggctgcaggaagaaccagtaccggcattattggagtgaaaaccttttccagtgcttcaattgcagcctctgcctcaatgggaccgtgcacctctcctgccaggagaaacagaacaccgtgtgcacctgccatgcaggtttctttctaagagaaaacgagtgtgtctcctgtagtaactgtaagaaaagcctggagtgcacgaagttgtgcctaccaagcttaggatccagatctaacttggggtggctttgtcttcttcttttgccaattccactaattgtttgggtgaagagaaaggaagtacagaaaacatgcagaaagcacagaaaggaaaaccaaggttctcatgaatctccaaccttaaatcctgaaacagtggcaataaatttatctgatgttgacttgagtaaatatatcaccactattgctggagtcatgacactaagtcaagttaaaggctttgttcgaaagaatggtgtcaatgaagccaaaatagatgagatcaagaatgacaatgtccaagacacagcagaacagaaagttcaactgcttcgtaattggcatcaacttcatggaaagaaagaagcgtatgacacattgattaaagatctcaaaaaagccaatctttgtactcttgcagagaaaattcagactatcatcctcaaggacattactagtgactcagaaaattcaaacttcagaaatgaaatccaaagcttggtctagctcgagcatgcatctaggcggccgcatggcagaaattcgcgaattcgctagcgttaacggatcctctagacgagatccgaacttgtttattgcagcttataatggttacaaataaagcaatagcatcacaaatttcacaaataaagcatttttttcactgcattctagttgtggtttgtccaaactcatcaatgtatcttatcatgtctagatctgtactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccggctgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgaacctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggcacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgccgttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaacagaaattaatcatgcagctgggagagtcctaaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcagccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggctagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactataacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgttgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatacgcgcccaccgaaaccgaattctcctggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccctggattacatcaagatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacgagtgcgtcaccggccgctgcaccacacctaccgcctgaccgtaaaccagactttttccggacagacctcaataactctgtttaccagaacaggaggtgagcttagaaaacccttagggtattaggccaaaggcgcagctactgtggggtttatgaacaattcaagcaactctacgggctattctaattcaggtttctctagaatcggggttggggttattctctgtcttgtgattctctttattcttatactaacgcttctctgcctaaggctcgccgcctgctgtgtgcacatttgcatttattgtcagctttttaaacgctggggtcgccacccaagatgattaggtacataatcctaggtttactcacccttgcgtcagcccacggtaccacccaaaaggtggattttaaggagccagcctgtaatgttacattcgcagctgaagctaatgagtgcaccactcttataaaatgcaccacagaacatgaaaagctgcttattcgccacaaaaacaaaattggcaagtatgctgtttatgctatttggcagccaggtgacactacagagtataatgttacagttttccagggtaaaagtcataaaacttttatgtatacttttccattttatgaaatgtgcgacattaccatgtacatgagcaaacagtataagttgtggcccccacaaaattgtgtggaaaacactggcactttctgctgcactgctatgctaattacagtgctcgctttggtctgtaccctactctatattaaatacaaaagcagacgcagctttattgaggaaaagaaaatgccttaatttactaagttacaaagctaatgtcaccactaactgctttactcgctgcttgcaaaacaaattcaaaaagttagcattataattagaataggatttaaaccccccggtcatttcctgctcaataccattcccctgaacaattgactctatgtgggatatgctccagcgctacaaccttgaagtcaggcttcctggatgtcagcatctgactttggccagcacctgtcccgcggatttgttccagtccaactacagcgacccaccctaacagagatgaccaacacaaccaacgcggccgccgctaccggacttacatctaccacaaatacaccccaagtttctgcctttgtcaataactgggataacttgggcatgtggtggttctccatagcgcttatgtttgtatgccttattattatgtggctcatctgctgcctaaagcgcaaacgcgcccgaccacccatctatagtcccatcattgtgctacacccaaacaatgatggaatccatagattggacggactgaaacacatgttcttttctcttacagtatgattaaatgagacatgattcctcgagtttttatattactgacccttgttgcgctttttttgtgcgtgctccacattggctgcggtttctcacatcgaagtagactgcattccagccttcacagtctatttgctttacggatttgtcaccctcacgctcatctgcagcctcatcactgtggtcatcgcctttatccagtgcattgactgggtctgtgtgcgctttgcatatctcagacaccatccccagtacagggacaggactatagctgagcttcttagaattctttaattatgaaatttactgtgacttttctgctgattatttgcaccctatctgcgttttgttccccgacctccaagcctcaaagacatatatcatgcagattcactcgtatatggaatattccaagttgctacaatgaaaaaagcgatctttccgaagcctggttatatgcaatcatctctgttatggtgttctgcagtaccatcttagccctagctatatatccctaccttgacattggctggaacgcaatagatgccatgaaccacccaactttccccgcgcccgctatgcttccactgcaacaagttgttgccggcggctttgtcccagccaatcagcctcgcccaccttctcccacccccactgaaatcagctactttaatctaacaggaggagatgactgacaccctagatctagaaatggacggaattattacagagcagcgcctgctagaaagacgcagggcagcggccgagcaacagcgcatgaatcaagagctccaagacatggttaacttgcaccagtgcaaaaggggtatcttttgtctggtaaagcaggccaaagtcacctacgacagtaataccaccggacaccgccttagctacaagttgccaaccaagcgtcagaaattggtggtcatggtgggagaaaagcccattaccataactcagcactcggtagaaaccgaaggctgcattcactcaccttgtcaaggacctgaggatctctgcacccttattaagaccctgtgcggtctcaaagatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatg

SEQ ID NO: 20-21 - skipped (2 entries)

SEQ ID NO: 22 - DNA, 19 bp, Artificial Sequence
  Endothelial Cell-Specific Enhancer Element
ggtgactttgcttctggag

SEQ ID NO: 23 - DNA, 19 bp, Artificial Sequence
  Endothelial Cell-Specific Enhancer Element
ctccagaagcaaagtcacc

SEQ ID NO: 24 - DNA, 19 bp, Artificial Sequence
  Endothelial Cell-Specific Enhancer Element
gtacttcatacttttcatt

SEQ ID NO: 25 - DNA, 19 bp, Artificial Sequence
  Endothelial Cell-Specific Enhancer Element
aatgaaaagtatgaagtac

SEQ ID NO: 26 - DNA, 6 bp, Artificial Sequence
  Hypoxia Response element
gcacgt

SEQ ID NO: 27 - protein, 40 aa, Artificial Sequence
  peptide linker
GGGSGGGSGGGSGGGSGGGSGGGSGGGSGGGSGGGSGGGS

SEQ ID NO: 28 - protein, 10 aa, Artificial Sequence
  VH CDR1
GYTFTNYGMN

SEQ ID NO: 29 - protein, 17 aa, Artificial Sequence
  VH CDR2
WINTYTGEPTYAADFKR

SEQ ID NO: 30 - protein, 14 aa, Artificial Sequence
  VH CDR3
YPHYYGSSHWYFDV

SEQ ID NO: 31 - protein, 11 aa, Artificial Sequence
  VL CDR1
SASQDISNYLN

SEQ ID NO: 32 - protein, 7 aa, Artificial Sequence
  VL CDR2
FTSSLHS

SEQ ID NO: 33 - protein, 9 aa, Artificial Sequence
  VL CDR3
QQYSTVPWT

(19) United States
(12) Patent Application Publication
(10) Pub. No.: US 2022/0154212 A1
(43) Pub. Date: May 19, 2022

(54) METHODS OF ANTI-TUMOR THERAPY

(71) Applicant: VASCULAR BIOGENICS LTD., Modi'in (IL)

(72) Inventors: Tamar RACHMILEWITZ MINEI, Modi'in (IL); Shifra FAIN-SHMUELI, Modi'in (IL)

(73) Assignee: VASCULAR BIOGENICS LTD., Modi'in (IL)

(21) Appl. No.: 17/438,840

(22) PCT Filed: Mar. 12, 2020
(86) PCT No.: PCT/IB2020/052260

§ 371 Date: Sep. 13, 2021

Related U.S. Application Data
(60) Provisional application No. 62/817,735, filed on Mar. 13, 2019.

(51) Int. Cl. C12N 15/86

(52) U.S. Cl. CPC C12N 15/86; A61K 48/00; C12N 2710/10343

(57) ABSTRACT

The invention provides methods for inducing or improving an anti-tumor response in a subject having a tumor, comprising administering to the subject a priming dose of a vector which comprises a Fas-chimera gene operably linked to an endothelial cell specific promoter, wherein the priming dose of the vector is administered prior to surgical removal of the tumor or a portion thereof.

### REFERENCE TO SEQUENCE LISTING SUBMITTED ELECTRONICALLY

The content of the electronically submitted sequence listing in ASCII text file (Name: 2020-03-13-SeqListing_ST25-3182-092PC01.txt; Size: 71.8 KB (73,613 bytes); and Date of Creation: Mar. 12, 2020) filed with the application is incorporated herein by reference in its entirety.

### REFERENCE TO RELATED APPLICATIONS

This application claims the benefit of U.S. Provisional Patent Application No. 62/817,735, filed on Mar. 13, 2019, incorporated herein by reference in its entirety.

### BACKGROUND OF THE DISCLOSURE

Angiogenesis is a common and major feature of several pathologies. Among these are diseases in which the angiogenesis can improve the disease condition (such as ischemic heart disease) and diseases in which the excessive angiogenesis is a part of the pathology and thus should be eliminated. These latter diseases include diabetes (diabetic retinopathy), cardiovascular diseases (atherosclerosis), chronic inflammation (rheumatoid arthritis), and cancer. Angiogenesis occurs in tumors and permits their growth, invasion and metastasis. In 1971, Folkman proposed that tumor growth and metastases are angiogenesis dependent, and thus inhibiting angiogenesis may be a strategy to arrest tumor growth.

There are several molecules involved in angiogenesis, from transcription factors to growth factors. Hypoxia is an important environmental factor that leads to neovascularization, and it induces release of several cytokines that are pro-angiogenic factors. Among them are vascular endothelial growth factors (VEGF) and their receptors, members of the angiopoietin family, basic fibroblast growth factor, and endothelin-1 (ET-1). These factors are involved in induction of angiogenesis through activation, proliferation and migration of endothelial cells.

Many chemotherapeutic agents have been tested as potential treatments for cancer, but these agents have been known for significant side effects. Recombinant forms of endogenous inhibitors of angiogenesis were also tested for the treatment of cancer. The potential pharmacokinetic, biotechnological and economic drawbacks of chronic delivery of these recombinant inhibitors have led scientists to develop other approaches.

The development of the anti-VEGF monoclonal antibody bevacizumab has validated an antiangiogenic approach as a complementary therapeutic modality to chemotherapy. Several small molecule inhibitors, including second-generation multi-targeted tyrosine kinase inhibitors, have also shown promise as antiangiogenic agents for cancer.

However, the potential pharmacokinetic and economic drawbacks of chronic delivery of recombinant inhibitors, antibodies, and small molecules, as well as the limited activity manifested when applied as monotherapy have led scientists to evaluate antiangiogenic gene therapy. Gene therapy is an emerging modality for treating inherited and acquired human diseases. However, there are a number of obstacles limiting successful gene therapy, including duration of expression, induction of the immune response, cytotoxicity of the vectors and tissue specificity. Two general strategies for the cancer gene therapy were proposed: tumor directed or systemic gene therapy. The lack of success in targeting gene therapy products to cancerous cells or their environment by systemic treatments caused most therapies to be administered to the tumor itself.

### BRIEF SUMMARY OF DISCLOSURE

The present disclosure is directed to a method for inducing or improving an anti-tumor response in a subject having a tumor, comprising administering to the subject a priming dose of a vector which comprises a Fas-chimera gene operably linked to an endothelial cell specific promoter, wherein the priming dose of the vector is administered prior to surgical removal of the tumor or a portion thereof, and wherein the anti-tumor response is induced or improved after the administration compared to the anti-tumor response in a subject not receiving a priming dose of the vector prior to surgical removal of the tumor or a portion thereof or in a subject not administered the vector.

The present disclosure further provides a method for treating a tumor in a subject in need thereof, comprising administering to the subject a priming dose of a vector which comprises a Fas-chimera gene operably linked to an endothelial cell specific promoter, wherein the priming dose of the vector is administered prior to surgical removal of the tumor or a portion thereof.

In some aspects, the methods of the present disclosure further comprise administering a post-surgical dose of the vector. In some aspects, the post-surgical dose of the vector is further administered in combination with one or more chemotherapeutic agents.

In some aspects of the disclosure, the one or more chemotherapeutic agents are selected from the group consisting of Acivicin; Aclarubicin; Acodazole Hydrochloride; Acronine; Adriamycin; Adozelesin; Aldesleukin; Alimta; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bevacizumab, Bizelesin; Bleomycin Sulfate; Brequinar Sodium; Bropirimine; Busulfan; Cactinomycin; Calusterone; Caracemide; Carbetimer; Carboplatin; Carmustine (BiCNU); Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflornithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Etoposide; Etoposide Phosphate; Etoprine; Fadrozole Hydrochloride; Fazarabine; Fenretinide; Floxuridine; Fludarabine Phosphate; Fluorouracil; Flurocitabine; Fosquidone; Fostriecin Sodium; Gemcitabine; Gemcitabine Hydrochloride; Gliadel® wafer; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-I a; Interferon Gamma-I b; Iproplatin; Irinotecan Hydrochloride; Lanreotide Acetate; Letrozole; Leuprolide Acetate; Liarozole Hydrochloride; Lometrexol Sodium; Lomustine (CCNU); Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; pazotinib; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safingol; Safingol Hydrochloride; Semustine; Simtrazene; Sorafinib; Sparfosate Sodium; Sparsomycin; Spirogermanium Hydrochloride; Spiromustine; Spiroplatin; Streptonigrin; Streptozocin; Sulofenur; Sunitinib; Talisomycin; Taxol; Tecogalan Sodium; Tegafur; Teloxantrone Hydrochloride; Temoporfin; Temozolomide; Teniposide; Teroxirone; Testolactone; Thiamiprine; Thioguanine; Thiotepa; Tiazofuirin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; and Zorubicin Hydrochloride.

In some aspects, the one or more chemotherapeutic agents are an anti-VEGF antibody or a VEGF binding molecule. In some aspects, the anti-VEGF antibody is a monoclonal antibody, a humanized antibody, a human antibody, a single chain antibody, or a chimeric antibody. In some aspects, the VEGF antagonist comprises Fab, F(ab)2, Fv, or scFv.

In some aspects, the one or more chemotherapeutic agents are an anti-VEGF receptor binding antibody or a VEGF receptor binding molecule. In some aspects, the anti-VEGF receptor binding antibody is a monoclonal antibody, a humanized antibody, a human antibody, a single chain antibody, or a chimeric antibody. In some aspects, the VEGF antagonist comprises Fab, F(ab)2, Fv, or scFv.

In a particular aspect, the VEGF antagonist is selected from the group consisting of bevacizumab, ranibizumab, VGX-100, r84, aflibercept, IMC-18F1, IMC-1C11, and ramucirumab. In a more particular aspect, the VEGF antagonist is bevacizumab.

In some aspects of the present disclosure, the Fas-chimera gene encodes a polypeptide comprising an extracellular domain of a TNF Receptor 1 (TNFR1) polypeptide fused to a transmembrane domain and an intracellular domain of a Fas polypeptide. In some aspects, the extracellular domain of the TNFR1 comprises an amino acid sequence at least 70%, 80%, 90%, 95%, 96%, 97%, 98%, 99%, or 100% identical to SEQ ID NO: 4, wherein the extracellular domain of the TNFR1 is capable of binding to TNF-α. In some aspects, the transmembrane domain and the intracellular domain of the Fas polypeptide comprise an amino acid sequence at least 70%, 80%, 90%, 95%, 96%, 97%, 98%, 99%, or 100% identical to SEQ ID NO: 8, wherein the transmembrane domain and the intracellular domain of the Fas polypeptide is capable of inducing Fas mediated apoptosis.

In some aspects of the disclosure, the Fas-chimera gene comprises a first nucleotide sequence, which is at least 70%, 80%, 90%, 95%, 96%, 97%, 98%, 99%, or 100% identical to SEQ ID NO: 3, and a second nucleotide sequence, which is at least 70%, 80%, 90%, 95%, 96%, 97%, 98%, 99%, or 100% identical to SEQ ID NO: 7.

In some aspects, the endothelial cell specific promoter comprises a PPE-1 promoter. In some aspects, the endothelial cell-specific promoter further comprises a cis-regulatory element comprising a sequence at least 70%, 80%, 90%, 95%, 96%, 97%, 98%, 99%, or 100% identical to SEQ ID NO: 15 or SEQ ID NO: 16, wherein the cis-regulatory element induces an improved endothelial cell specificity compared to an endothelial cell specific promoter without the cis-regulatory element. In particular aspects, the cis-regulatory element comprises SEQ ID NO: 11 or SEQ ID NO: 12. In some aspects, the cis-regulatory element further comprises SEQ ID NO: 13 or SEQ ID NO: 14.

In some aspects, the endothelial cell specific promoter is a PPE-1-3X promoter. In particular aspects, the PPE-1-3X promoter comprises a nucleotide sequence at least 70%, 80%, 90%, 95%, 96%, 97%, 98%, 99%, or 100% identical to SEQ ID NO: 18, wherein the PPE-1-3X promoter is capable of directing the Fas-chimera gene expression in endothelial cells.

In some aspects of the present disclosure, the vector does not contain an E1 region of an adenovirus.

In some aspects, the priming dose of the vector is administered about 7 days, about 8 days, about 9 days, about 10 days, about 11 days, about 12 days, about 13 days, about 2 weeks, about 15 days, about 20 days, about 3 weeks, about 25 days, about 4 weeks, about a month, about 5 weeks, about 6 weeks, about 7 weeks, about 2 months, about 3 months, about 4 months, about 5 months, or about 6 months before surgical removal of the tumor or a portion thereof.

In some aspects, the post-surgical dose of the vector and the one or more chemotherapeutic agents are administered sequentially. In some aspects, the post-surgical dose of the vector is administered prior to the one or more chemotherapeutic agents.

In some aspects of the present disclosure, the priming dose of the vector is administered at an effective amount of less than about 1×10¹⁵, less than about 1×10¹⁴, less than about 5×10¹³, less than about 4×10¹³, less than about 3×10¹³, less than about 2×10¹³, less than about 1×10¹³, less than about 9×10¹², less than about 8×10¹², less than about 7×10¹², less than about 6×10¹², less than about 5×10¹², less than about 4×10¹², less than about 3×10¹², less than about 2×10¹², less than about 1×10¹², less than about 9×10¹¹, less than about 8×10¹¹, less than about 7×10¹¹, less than about 6×10¹¹, less than about 5×10¹¹, less than about 4×10¹¹, less than about 3×10¹¹, less than about 2×10¹¹, less than about 1×10¹¹, less than about 9×10¹⁰, less than about 8×10¹⁰, less than about 7×10¹⁰, less than about 6×10¹⁰, less than about 5×10¹⁰, less than about 4×10¹⁰, less than about 3×10¹⁰, less than about 2×10¹⁰, or less than about 1×10¹⁰ virus particles.

In some aspects, the post-surgical dose of the vector is administered at an effective amount of less than about 1×10¹⁵, less than about 1×10¹⁴, less than about 5×10¹³, less than about 4×10¹³, less than about 3×10¹³, less than about 2×10¹³, less than about 1×10¹³, less than about 9×10¹², less than about 8×10¹², less than about 7×10¹², less than about 6×10¹², less than about 5×10¹², less than about 4×10¹², less than about 3×10¹², less than about 2×10¹², less than about 1×10¹², less than about 9×10¹¹, less than about 8×10¹¹, less than about 7×10¹¹, less than about 6×10¹¹, less than about 5×10¹¹, less than about 4×10¹¹, less than about 3×10¹¹, less than about 2×10¹¹, less than about 1×10¹¹, less than about 9×10¹⁰, less than about 8×10¹⁰, less than about 7×10¹⁰, less than about 6×10¹⁰, less than about 5×10¹⁰, less than about 4×10¹⁰, less than about 3×10¹⁰ less than about 2×10¹⁰, or less than about 1×10¹⁰ virus particles.

In some aspects of the disclosure, the bevacizumab is administered at an effective amount of less than about 15 mg/kg, 14 mg/kg, 13 mg/kg, 12 mg/kg, 11 mg/kg, 10 mg/kg, 9 mg/kg, 8 mg/kg, 7 mg/kg, 6 mg/kg, 5 mg/kg, 4 mg/kg, 3 mg/kg, 2 mg/kg, or 1 mg/kg.

In particular aspects, the post-surgical dose of the vector is administered at an effective amount of 3×10¹² to 3×10¹³ virus particles and bevacizumab is administered at an effective amount of 5 mg/kg to 15 mg/kg.

In some aspects of the present disclosure, the post-surgical dose of the vector is repeatedly administered.

In some aspects, the priming dose of the vector and the post-surgical dose of the vector are the same. In other aspects, the priming dose of the vector and the post-surgical dose of the vector are different. In some aspects, wherein the priming dose of the vector is higher than the post-surgical dose of the vector. In other aspects, the priming dose of the vector is lower than the post-surgical dose of the vector.

In some aspects of the present disclosure, the post-surgical dose of the vector is repeatedly administered every day, once in about 2 days, once in about 3 days, once in about 4 days, once in about 5 days, once in about 6 days, once in about 7 days, once in about 2 weeks, once in about 3 weeks, once in about 4 weeks, once in about 5 weeks, once in about 6 weeks, once in about 7 weeks, once in about 2 months, or once in about 6 months.

In some aspects, the bevacizumab is repeatedly administered. In particular aspects, the bevacizumab is repeatedly administered once in about 7 days, once in about 2 weeks, once in about 3 weeks, once in about 4 weeks, once in about 2 months, once in about 3 months, once in about 4 months, once in about 5 months, or once in about 6 months.

In more particular aspects, the post-surgical dose of the vector is administered every 2 months and bevacizumab is administered every 2 weeks.

In some aspects of the present disclosure, the tumor is derived from or associated with a sarcoma, melanoma, carcinoma, leukemia, Hodgkin's Disease, Non-Hodgkin's Lymphoma, multiple myeloma, neuroblastoma, breast cancer, ovarian cancer, lung cancer (including non-small cell lung cancer (NSCLC)), rhabdomyosarcoma, primary thrombocytosis, primary macroglobulinemia, small-cell lung tumors, primary brain tumors, gliomas (including glioblastoma multiforme (GBM) and recurrent GBM), stomach cancer, colon cancer (including metastatic colorectal cancer (mCRC)), hepatobiliary cancer, malignant pancreatic insulanoma, malignant carcinoid, urinary bladder cancer, premalignant skin lesions, testicular cancer, lymphomas, thyroid cancer, papillary thyroid cancer, neuroblastoma, neuroendocrine cancer, esophageal cancer, genitourinary tract cancer, malignant hypercalcemia, cervical cancer, endometrial cancer, adrenal cortical cancer, prostate cancer, Müllerian cancer, ovarian cancer, peritoneal cancer, fallopian tube cancer, or uterine papillary serous carcinoma.

In some aspects, the sarcoma is chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

In some aspects, the melanoma is acra-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, metastatic melanoma, nodular melanoma, subungal melanoma, or superficial spreading melanoma.

In some aspects, the carcinoma is acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniform carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidernoid carcinoma, carcinoma mucosum, mucous carcinoma, carcinoma myxomatodes, naspharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma viflosum.

In particular aspects, the tumor is derived from or associated with glioblastoma. In a more particular aspect, the GBM is a recurrent GBM.

In some aspects, the vector is an adenovirus vector. In particular aspects, the adenovirus vector is adenovirus serotype 5. In more particular aspects, the vector comprises, consists of, or consists essentially of SEQ ID NO: 19. In more particular aspects, the vector is an isolated virus having European Collection of Cell Cultures (ECACC) Accession Number 13021201.

### EMBODIMENTS

1. A method for inducing or improving an anti-tumor response in a subject having a tumor, comprising administering to the subject a priming dose of a vector which comprises a Fas-chimera gene operably linked to an endothelial cell specific promoter, wherein the priming dose of the vector is administered prior to surgical removal of the tumor or a portion thereof, and wherein the anti-tumor response is induced or improved after the administration compared to the anti-tumor response in a subject not receiving a priming dose of the vector prior to surgical removal of the tumor or a portion thereof or in a subject not administered the vector.

2. A method for treating a tumor in a subject in need thereof, comprising administering to the subject a priming dose of a vector which comprises a Fas-chimera gene operably linked to an endothelial cell specific promoter, wherein the priming dose of the vector is administered prior to surgical removal of the tumor or a portion thereof.

3. The method of embodiment 1 or 2, further comprising administering a post-surgical dose of the vector.

4. The method of embodiment 3, wherein the post-surgical dose of the vector is further administered in combination with one or more chemotherapeutic agents.

5. The method of embodiment 4, wherein the one or more chemotherapeutic agents is selected from the group consisting of Acivicin; Aclarubicin; Acodazole Hydrochloride; Acronine; Adriamycin; Adozelesin; Aldesleukin; Alimta; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bevacizumab, Bizelesin; Bleomycin Sulfate; Brequinar Sodium; Bropirimine; Busulfan; Cactinomycin; Calusterone; Caracemide; Carbetimer; Carboplatin; Carmustine (BiCNU); Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflornithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Etoposide; Etoposide Phosphate; Etoprine; Fadrozole Hydrochloride; Fazarabine; Fenretinide; Floxuridine; Fludarabine Phosphate; Fluorouracil; Flurocitabine; Fosquidone; Fostriecin Sodium; Gemcitabine; Gemcitabine Hydrochloride; Gliadel® wafer; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-I a; Interferon Gamma-I b; Iproplatin; Irinotecan Hydrochloride; Lanreotide Acetate; Letrozole; Leuprolide Acetate; Liarozole Hydrochloride; Lometrexol Sodium; Lomustine (CCNU); Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; pazotinib; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safingol; Safingol Hydrochloride; Semustine; Simtrazene; Sorafinib; Sparfosate Sodium; Sparsomycin; Spirogermanium Hydrochloride; Spiromustine; Spiroplatin; Streptonigrin; Streptozocin; Sulofenur; Sunitinib; Talisomycin; Taxol; Tecogalan Sodium; Tegafur; Teloxantrone Hydrochloride; Temoporfin; Temozolomide; Teniposide; Teroxirone; Testolactone; Thiamiprine; Thioguanine; Thiotepa; Tiazofuirin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; and Zorubicin Hydrochloride.

6. The method of embodiment 4, wherein the one or more chemotherapeutic agents is an anti-VEGF antibody or a VEGF binding molecule.

7. The method of embodiment 6, wherein the anti-VEGF antibody is a monoclonal antibody, a humanized antibody, a human antibody, a single chain antibody, or a chimeric antibody.

8. The method of embodiment 6 or 7, wherein the VEGF antagonist comprises Fab, F(ab)₂, Fv, or scFv.

9. The method of embodiment 4, wherein the one or more chemotherapeutic agents is an anti-VEGF receptor binding antibody or a VEGF receptor binding molecule.

10. The method of embodiment 9, wherein the anti-VEGF receptor binding antibody is a monoclonal antibody, a humanized antibody, a human antibody, a single chain antibody, or a chimeric antibody.

11. The method of embodiment 9 or 10 wherein the VEGF antagonist comprises Fab, F(ab)₂, Fv, or scFv

12. The method of any one of embodiments 6 to 11, wherein the VEGF antagonist is selected from the group consisting of bevacizumab, ranibizumab, VGX-100, r84, aflibercept, IMC-18F1, IMC-1C11, and ramucirumab.

13. The method of embodiment 12, wherein the VEGF antagonist is bevacizumab.

14. The method of any one of embodiments 1 to 13, wherein the Fas-chimera gene encodes a polypeptide comprising an extracellular domain of a TNF Receptor 1 (TNFR1) polypeptide fused to a transmembrane domain and an intracellular domain of a Fas polypeptide.

15. The method of embodiment 14, wherein the extracellular domain of the TNFR1 comprises an amino acid sequence at least 70%, 80%, 90%, 95%, 96%, 97%, 98%, 99%, or 100% identical to SEQ ID NO: 4, wherein the extracellular domain of the TNFR1 is capable of binding to TNF-α.

16. The method of embodiment 14 or 15, wherein the transmembrane domain and the intracellular domain of the Fas polypeptide comprise an amino acid sequence at least 70%, 80%, 90%, 95%, 96%, 97%, 98%, 99%, or 100% identical to SEQ ID NO: 8, wherein the transmembrane domain and the intracellular domain of the Fas polypeptide is capable of inducing Fas mediated apoptosis.

17. The method of any of embodiments 1 to 16, wherein the Fas-chimera gene comprises a first nucleotide sequence, which is at least 70%, 80%, 90%, 95%, 96%, 97%, 98%, 99%, or 100% identical to SEQ ID NO: 3, and a second nucleotide sequence, which is at least 70%, 80%, 90%, 95%, 96%, 97%, 98%, 99%, or 100% identical to SEQ ID NO: 7.

18. The method of any one of embodiments 1 to 17, wherein the endothelial cell specific promoter comprises a PPE-1 promoter.

19. The method of any one of embodiments 1 to 17, wherein the endothelial cell-specific promoter further comprises a cis-regulatory element comprising a sequence at least 70%, 80%, 90%, 95%, 96%, 97%, 98%, 99%, or 100% identical to SEQ ID NO: 15 or SEQ ID NO: 16, wherein the cis-regulatory element induces an improved endothelial cell specificity compared to an endothelial cell specific promoter without the cis-regulatory element.

20. The method of embodiment 19, wherein the cis-regulatory element comprises SEQ ID NO: 11 or SEQ ID NO: 12.

21. The method of embodiment 20, wherein the cis-regulatory element further comprises SEQ ID NO: 13 or SEQ ID NO: 14.

22. The method of any one of embodiments 1 to 21, wherein the endothelial cell specific promoter is a PPE-1-3X promoter.

23. The method of embodiment 22, wherein the PPE-1-3X promoter comprises a nucleotide sequence at least 70%, 80%, 90%, 95%, 96%, 97%, 98%, 99%, or 100% identical to SEQ ID NO: 18, wherein the PPE-1-3X promoter is capable of directing the Fas-chimera gene expression in endothelial cells.

24. The method of any one of embodiments 1 to 23, wherein the vector does not contain an E1 region of an adenovirus.

25. The method of any one of embodiments 1 to 24, wherein the priming dose of the vector is administered about 7 days, about 8 days, about 9 days, about 10 days, about 11 days, about 12 days, about 13 days, about 2 weeks, about 15 days, about 20 days, about 3 weeks, about 25 days, about 4 weeks, about a month, about 5 weeks, about 6 weeks, about 7 weeks, about 2 months, about 3 months, about 4 months, about 5 months, or about 6 months before surgical removal of the tumor or a portion thereof.

26. The method of any one of embodiments 3 to 25, wherein the post-surgical dose of the vector and the one or more chemotherapeutic agents are administered sequentially.

27. The method of embodiment 26, wherein the post-surgical dose of the vector is administered prior to the one or more chemotherapeutic agents.

28. The method of any one of embodiments 1 to 27, wherein the priming dose of the vector is administered at an effective amount of less than about 1×10¹⁵, less than about 1×10¹⁴, less than about 5×10¹³, less than about 4×10¹³, less than about 3×10¹³, less than about 2×10¹³, less than about 1×10¹³, less than about 9×10¹², less than about 8×10¹², less than about 7×10¹², less than about 6×10¹², less than about 5×10¹², less than about 4×10¹², less than about 3×10¹², less than about 2×10¹², less than about 1×10¹², less than about 9×10¹¹, less than about 8×10¹¹, less than about 7×10¹¹, less than about 6×10¹¹, less than about 5×10¹¹, less than about 4×10¹¹, less than about 3×10¹¹, less than about 2×10¹¹, less than about 1×10¹¹, less than about 9×10¹⁰, less than about 8×10¹⁰, less than about 7×10¹⁰, less than about 6×10¹⁰, less than about 5×10¹⁰, less than about 4×10¹⁰, less than about 3×10¹⁰, less than about 2×10¹⁰, or less than about 1×10¹⁰ virus particles.

29. The method of any one of embodiments 3 to 28, wherein the post-surgical dose of the vector is administered at an effective amount of less than about 1×10¹⁵, less than about 1×10¹⁴, less than about 5×10¹³, less than about 4×10¹³, less than about 3×10¹³, less than about 2×10¹³, less than about 1×10¹³, less than about 9×10¹², less than about 8×10¹², less than about 7×10¹², less than about 6×10¹², less than about 5×10¹², less than about 4×10¹², less than about 3×10¹², less than about 2×10¹², less than about 1×10¹², less than about 9×10¹¹, less than about 8×10¹¹, less than about 7×10¹¹, less than about 6×10¹¹, less than about 5×10¹¹, less than about 4×10¹¹, less than about 3×10¹¹, less than about 2×10¹¹, less than about 1×10¹¹, less than about 9×10¹⁰, less than about 8×10¹⁰, less than about 7×10¹⁰, less than about 6×10¹⁰, less than about 5×10¹⁰, less than about 4×10¹⁰, less than about 3×10¹⁰, less than about 2×10¹⁰, or less than about 1×10¹⁰ virus particles.

30. The method of any one of embodiments 12 to 29, wherein bevacizumab is administered at an effective amount of less than about 15 mg/kg, 14 mg/kg, 13 mg/kg, 12 mg/kg, 11 mg/kg, 10 mg/kg, 9 mg/kg, 8 mg/kg, 7 mg/kg, 6 mg/kg, 5 mg/kg, 4 mg/kg, 3 mg/kg, 2 mg/kg, or 1 mg/kg.

31. The method of any one of embodiments 12 to 29, wherein the post-surgical dose of the vector is administered at an effective amount of 3×10¹² to 3×10¹³ virus particles and bevacizumab is administered at an effective amount of 5 mg/kg to 15 mg/kg.

32. The method of any one of embodiments 3 to 31, wherein the post-surgical dose of the vector is repeatedly administered.

33. The method of any one of embodiments 3 to 32, wherein the priming dose of the vector and the post-surgical dose of the vector are the same.

34. The method of any one of embodiments 3 to 32, wherein the priming dose of the vector and the post-surgical dose of the vector are different.

35. The method of any one of embodiments 3 to 32, wherein the priming dose of the vector is higher than the post-surgical dose of the vector.

36. The method of any one of embodiments 3 to 32, wherein the priming dose of the vector is lower than the post-surgical dose of the vector.

37. The method of any one of embodiments 32 to 36, wherein the post-surgical dose of the vector is repeatedly administered every day, once in about 2 days, once in about 3 days, once in about 4 days, once in about 5 days, once in about 6 days, once in about 7 days, once in about 2 weeks, once in about 3 weeks, once in about 4 weeks, once in about 5 weeks, once in about 6 weeks, once in about 7 weeks, once in about 2 months, or once in about 6 months.

38. The method of any one of embodiments 12 to 37, wherein the bevacizumab is repeatedly administered.

39. The method of embodiment 38, wherein the bevacizumab is repeatedly administered once in about 7 days, once in about 2 weeks, once in about 3 weeks, once in about 4 weeks, once in about 2 months, once in about 3 months, once in about 4 months, once in about 5 months, or once in about 6 months.

40. The method of any one of embodiments 12 to 39, wherein the post-surgical dose of the vector is administered every 2 months and bevacizumab is administered every 2 weeks.

41. The method of any one of embodiments 1 to 40, wherein the tumor is derived from or associated with a sarcoma, melanoma, carcinoma, leukemia, Hodgkin's Disease, Non-Hodgkin's Lymphoma, multiple myeloma, neuroblastoma, breast cancer, ovarian cancer, lung cancer (including non-small cell lung cancer (NSCLC)), rhabdomyosarcoma, primary thrombocytosis, primary macroglobulinemia, small-cell lung tumors, primary brain tumors, gliomas (including glioblastoma multiforme (GBM) and recurrent GBM), stomach cancer, colon cancer (including metastatic colorectal cancer (mCRC)), hepatobiliary cancer, malignant pancreatic insulanoma, malignant carcinoid, urinary bladder cancer, premalignant skin lesions, testicular cancer, lymphomas, thyroid cancer, papillary thyroid cancer, neuroblastoma, neuroendocrine cancer, esophageal cancer, genitourinary tract cancer, malignant hypercalcemia, cervical cancer, endometrial cancer, adrenal cortical cancer, prostate cancer, Müllerian cancer, ovarian cancer, peritoneal cancer, fallopian tube cancer, or uterine papillary serous carcinoma.

42. The method of embodiment 41, wherein the sarcoma is chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

43. The method of embodiment 41, wherein the melanoma is acra-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, metastatic melanoma, nodular melanoma, subungal melanoma, or superficial spreading melanoma.

44. The method of embodiment 41, wherein the carcinoma is acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniform carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidernoid carcinoma, carcinoma mucosum, mucous carcinoma, carcinoma myxomatodes, naspharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma viflosum.

45. The method of any one of embodiments 1 to 41, wherein the tumor is derived from or associated with glioblastoma.

46. The method of embodiment 45, wherein the glioblastoma is a recurrent glioblastoma.

47. The method of any one of embodiments 1 to 46, wherein the vector is an adenovirus vector.

48. The method of embodiment 47 wherein the adenovirus vector is adenovirus serotype 5.

49. The method of any one of embodiments 1 to 48, wherein the vector comprises, consists of, or consists essentially of SEQ ID NO: 19.

50. The method of any one of embodiments 1 to 49, wherein the vector is an isolated virus having European Collection of Cell Cultures (ECACC) Accession Number 13021201.

### BRIEF DESCRIPTION OF THE DRAWINGS/FIGURES

FIG. 1 shows a neoadjuvant therapy regimen for an Ad5-PPE-1-3X-Fas-c vector (e.g., VB-111). Patients in Group A receive intravenous infusion of VB-111 at 1×10¹³ VPs 21±7 days prior to surgical removal of the tumor. Patients in Groups B and C receive intravenous infusion of placebo 21±7 days prior to surgical removal of the tumor. After recovery from surgery, patients in Groups A and B receive intravenous infusion of VB-111 at 1×10¹³ VPs every 8 weeks and patients in Group C receive standard of care every 8 weeks. Upon disease progression, patients in Groups A and B may also receive infusions of bevacizumab if clinically indicated. XRT, radiation therapy; TMZ, temozolomide; PFS, progression-free survival; OS, overall survival; SOC, standard of care.

### DETAILED DESCRIPTION OF THE INVENTION

### I. Definitions

Unless defined otherwise, all technical and scientific terms used herein have the same meaning as commonly understood by one of ordinary skill in the art to which this invention belongs. In case of conflict, the present application including the definitions will control. Unless otherwise required by context, singular terms shall include pluralities and plural terms shall include the singular. All publications, patents and other references mentioned herein are incorporated by reference in their entireties for all purposes as if each individual publication or patent application were specifically and individually indicated to be incorporated by reference.

Although methods and materials similar or equivalent to those described herein can be used in practice or testing of the present disclosure, suitable methods and materials are described below. The materials, methods and examples are illustrative only and are not intended to be limiting. Other features and advantages of the invention will be apparent from the detailed description and from the claims.

In order to further define this invention, the following terms and definitions are provided.

Throughout this disclosure, the term “a” or “an” entity refers to one or more of that entity; for example, “a polynucleotide,” is understood to represent one or more polynucleotides. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. Unless otherwise indicated, amino acid sequences are written left to right in amino to carboxy orientation. The headings provided herein are not limitations of the various aspects of the disclosure, which can be had by reference to the specification as a whole. Accordingly, the terms defined immediately below are more fully defined by reference to the specification in its entirety.

The term “about” is used herein to mean approximately, roughly, around, or in the regions of. When the term “about” is used in conjunction with a numerical range, it modifies that range by extending the boundaries above and below the numerical values set forth. In general, the term “about” is used herein to modify a numerical value above and below the stated value by a variance of 10 percent, up or down (higher or lower).

As used herein, “antibody” means an intact immunoglobulin, an antigen-binding fragment thereof, or an antigen-binding molecule. Antibodies of this invention can be of any isotype or class (e.g., M, D, G, E and A) or any subclass (e.g., G1-4, A1-2) and can have either a kappa (κ) or lambda (λ) light chain.

The term “effective amount” as used herein refers to an amount effective, at dosages and for periods of time necessary, to achieve a desired result. A desired result can be, for example, eliciting an anti-tumor response or reduction or inhibition of neo-vascularization or angiogenesis in vitro or in vivo. An effective amount need not be a “cure” or complete removal of neo-vascularization or angiogenesis. In some embodiments, an effective amount can reduce a size or volume of a tumor. In other embodiments, an effective amount can reduce or ameliorate one or more symptoms of a cancer.

The term “polynucleotide” or “nucleotide” is intended to encompass a singular nucleic acid as well as plural nucleic acids, and refers to an isolated nucleic acid molecule or construct, e.g., messenger RNA (mRNA) or plasmid DNA (pDNA). In certain embodiments, a polynucleotide comprises a conventional phosphodiester bond or a non-conventional bond (e.g., an amide bond, such as found in peptide nucleic acids (PNA)).

As used herein, a “polynucleotide,” “nucleotide,” or “nucleic acid” can be used interchangeably and contain the nucleotide sequence of the full-length cDNA sequence, including the untranslated 5′ and 3′ sequences, the coding sequences, as well as fragments, epitopes, domains, and variants of the nucleic acid sequence. The polynucleotide can be composed of any polyribonucleotide or polydeoxyribonucleotide, which may be unmodified RNA or DNA or modified RNA or DNA. For example, polynucleotides can be composed of single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, the polynucleotides can be composed of triple-stranded regions comprising RNA or DNA or both RNA and DNA. Polynucleotides may also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms.

In the present disclosure, a polypeptide can be composed of amino acids joined to each other by peptide bonds or modified peptide bonds, i.e., peptide isosteres, and may contain amino acids other than the 20 gene-encoded amino acids (e.g. non-naturally occurring amino acids). The polypeptides of the present disclosure may be modified by either natural process, such as posttranslational processing, or by chemical modification techniques which are well known in the art. Such modifications are well described in basic texts and in more detailed monographs, as well as in a voluminous research literature. Modifications can occur anywhere in the polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched, for example, as a result of ubiquitination, and they may be cyclic, with or without branching. Cyclic, branched, and branched cyclic polypeptides may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. (See, for instance, Proteins—Structure And Molecular Properties, 2nd Ed., T. E. Creighton, W. H. Freeman and Company, New York (1993); Posttranslational Covalent Modification of Proteins, B. C. Johnson, Ed., Academic Press, New York, pgs. 1-12 (1983); Seifter et al., Meth Enzymol 182:626-646 (1990); Rattan et al., Ann NY Acad Sci 663:48-62 (1992).)

The terms “fragment,” “variant,” “derivative” and “analog” when referring to any polypeptide or polynucleotide of the present disclosure include any polypeptides or polynucleotides which retain at least some activities, i.e., the ability to function as any naturally-occurring function of the polypeptide or polynucleotide. For example, a “fragment,” “variant,” “derivative” and “analog” of Tumor necrosis factor Receptor 1 (TNFR1) has some activities of the naturally occurring full-length TNFR1, e.g., the ability to bind to TNFR1 ligand, i.e., TNF-alpha or lymphotoxin. In another example, a “fragment,” “variant,” “derivative” and “analog” of a Fas polypeptide have some activities of a naturally-occurring full-length Fas polypeptide, e.g., the ability to induce apoptosis. In other examples, a “fragment,” “variant,” “derivative” and “analog” of an endothelial cell-specific promoter can induce endothelial cell-specific expression of a gene operably linked to the promoter. Additional non-limiting examples of the various fragments, variants, analogues, or derivatives of the TNFR1, Fas polypeptide, and endothelial cell-specific promoters are described below.

The term “percent sequence identity” between two polynucleotide or polypeptide sequences refers to the number of identical matched positions shared by the sequences over a comparison window, taking into account additions or deletions (i.e., gaps) that must be introduced for optimal alignment of the two sequences. A matched position is any position where an identical nucleotide or amino acid is presented in both the target and reference sequence. Gaps presented in the target sequence are not counted since gaps are not nucleotides or amino acids. Likewise, gaps presented in the reference sequence are not counted since target sequence nucleotides or amino acids are counted, not nucleotides or amino acids from the reference sequence.

The percentage of sequence identity is calculated by determining the number of positions at which the identical amino-acid residue or nucleic acid base occurs in both sequences to yield the number of matched positions, dividing the number of matched positions by the total number of positions in the window of comparison and multiplying the result by 100 to yield the percentage of sequence identity. The comparison of sequences and determination of percent sequence identity between two sequences may be accomplished using readily available software both for online use and for download. Suitable software programs are available from various sources, and for alignment of both protein and nucleotide sequences. One suitable program to determine percent sequence identity is bl2seq, part of the BLAST suite of program available from the U.S. government's National Center for Biotechnology Information BLAST web site (blast.ncbi.nlm.nih.gov). Bl2seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the European Bioinformatics Institute (EBI) at www.ebi.ac.uk/Tools/psa.

Different regions within a single polynucleotide or polypeptide target sequence that aligns with a polynucleotide or polypeptide reference sequence can each have their own percent sequence identity. It is noted that the percent sequence identity value is rounded to the nearest tenth. For example, 80.11, 80.12, 80.13, and 80.14 are rounded down to 80.1, while 80.15, 80.16, 80.17, 80.18, and 80.19 are rounded up to 80.2. It also is noted that the length value will always be an integer.

One skilled in the art will appreciate that the generation of a sequence alignment for the calculation of a percent sequence identity is not limited to binary sequence-sequence comparisons exclusively driven by primary sequence data. Sequence alignments can be derived from multiple sequence alignments. One suitable program to generate multiple sequence alignments is ClustalW2, available from www.clustal.org. Another suitable program is MUSCLE, available from www.drive5.com/muscle/. ClustalW2 and MUSCLE are alternatively available, e.g., from the EBI.

It will also be appreciated that sequence alignments can be generated by integrating sequence data with data from heterogeneous sources such as structural data (e.g., crystallographic protein structures), functional data (e.g., location of mutations), or phylogenetic data. A suitable program that integrates heterogeneous data to generate a multiple sequence alignment is T-Coffee, available at www.tcoffee.org, and alternatively available, e.g., from the EBI. It will also be appreciated that the final alignment used to calculated percent sequence identity may be curated either automatically or manually.

As used herein, the terms “linked,” “fused,” “fusion,” “chimeric,” and “chimera” are used interchangeably. These terms refer to the joining together of two more elements or components, by whatever means including chemical conjugation or recombinant means. An “in-frame fusion” refers to the joining of two or more open reading frames (ORFs) to form a continuous longer ORF, in a manner that maintains the correct reading frame of the original ORFs. Thus, the resulting recombinant fusion or chimeric protein is a single protein containing two or more segments that correspond to polypeptides encoded by the original ORFs (which segments are not normally so joined in nature.) Although the reading frame is thus made continuous throughout the fused segments, the segments may be physically or spatially separated by, for example, in-frame linker sequence.

The term “heterologous nucleotide sequence” means that a polynucleotide is derived from a distinct entity from that of the entity to which it is being compared. For instance, a heterologous polynucleotide can be synthetic, or derived from a different species, different cell type of an individual, or the same or different type of cell of distinct individuals. In one aspect, a heterologous nucleotide sequence can be a polynucleotide operably linked to another polynucleotide to produce a fusion polynucleotide. In some aspects, a heterologous nucleotide sequence can encode a polypeptide. For example, a heterologous nucleotide sequence can be a promoter element operably linked to a gene encoding a polypetide. A heterologous nucleotide sequence can also include other cis-regulatory elements, such as enhancers, silencers, or transcription factors, operably linked to a gene encoding a polypeptide. In other aspects, a heterologous nucleotide sequence does not encode a polypeptide.

The term “expression” as used herein refers to a process by which a gene produces a biochemical, for example, an RNA or polypeptide. The process includes any manifestation of the functional presence of the gene within the cell including, without limitation, gene knockdown as well as both transient expression and stable expression. It includes without limitation transcription of the gene into messenger RNA (mRNA), transfer RNA (tRNA), small hairpin RNA (shRNA), small interfering RNA (siRNA) or any other RNA product and the translation of such mRNA into polypeptide(s). If the final desired product is biochemical, expression includes the creation of that biochemical and any precursors.

The term “complementarity determining region” (CDR) as used herein refers to the amino acid residues of an antibody which are responsible for binding to an antigen. The CDR regions of an antibody are found within the hypervariable region of both heavy and light chains of the antibody. Full length antibodies comprise three CDR regions in the heavy chain variable domain and three CDR regions in the light chain variable domain.

The term “surgical removal” as used herein refers to surgical removal of a tumor or a portion thereof from a subject for purposes of alleviating tumor burden in the subject. Surgical removal of a tumor can be a complete or partial tumor resection. Surgical removal of a tumor or a portion thereof can be performed using standard, accepted medical procedures in the art. In the present disclosure, “surgical removal” of a tumor or a portion thereof does not include removal of tumor tissue for biopsy or diagnostic purposes.

The term “priming dose” as used herein refers to administration of a vector to a subject having a tumor prior to surgical removal of the tumor or a portion thereof. The term “post-surgical dose” as used herein refers to administration of a vector to a subject after the subject has had surgical removal of a tumor or a portion thereof.

The term “anti-tumor response” as used herein refers to a subject's bodily response against the presence of a tumor. For example, in some aspects the anti-tumor response in the present disclosure can be an anti-tumor immune response. In some aspects, an anti-tumor immune response is characterized by the presence of tumor-infiltrating CD8⁺ lymphocytes within the tumor bed. In some aspects, an anti-tumor immune response is characterized by a particular cytokine profile in the subject. In some aspects, an anti-tumor immune response is characterized by the presence of circulating anti-tumor antibodies in the subject directed against tumor markers or tumor tissue.

The term “repeatedly administered” as used herein refers to administration of a therapeutic agent on a repeated basis at defined, fixed intervals. The intervals of time between each administration may be altered during the course of the repeated administration and may be as long as 1 day, 2 days, 3 days, 4 days, 5 days, 6 days, 7 days, 2 weeks, 3 weeks, 4 weeks, 5 weeks, 6 weeks, 7 weeks, 2 months, 3 months, 4 months, 5 months, 6 months, 7 months, 8 months, 9 months, 10 months, 11 months, 1 year, or more.

The term “combination therapy” as used herein refers to the administration of two or more therapeutic modalities to treat a disease or condition. In some aspects of the disclosure, combination therapy refers to administration of a vector and one or more chemotherapeutic agents to a subject in need thereof. In some embodiments, the combination therapy comprises administering the one or more chemotherapeutic agents prior to administering the vector. In another embodiment, the combination therapy comprises administering the one or more chemotherapeutic agents concomitantly with administration of the vector. In another embodiment, the combination therapy comprises administering the one or more chemotherapeutic agents after administering the vector. In some aspects, the vector and the one or more chemotherapeutic agents are administered as a combination therapy to a subject having a tumor after surgical removal of the tumor or a portion thereof. In some aspects, the one or more chemotherapeutic agents is a VEG antagonist.

The term “adenovirus” as used herein refers to a human adenovirus of the Adenoviridae family. An adenovirus of the present disclosure can include, for example, an adenovirus from any one of seven species and 57 serotypes, including species A (serotypes 12, 18, and 31), species B (serotypes 3, 7, 11, 14, 16, 21, 34, 35, 50, and 55), species C (serotypes 1, 2, 5, 6, and 57), species D (8, 9, 10, 13, 15, 17, 19, 20, 22-30, 32, 33, 36-39, 42-49, 51, 53, 54, and 56), species E (serotype 4), species F (serotype 40 and 41), or species G (serotype 52). The term “adenovirus vector” as used herein refers to an adenovirus that has been genetically modified to behave differently from the natural wildtype virus. For example, an adenovirus vector may be modified so that it is unable to replicate outside of a particular packaging cell line. In some aspects, an adenovirus vector is genetically modified to carry one or more genes encoding non-adenoviral proteins.

### II. Treatment Methods of the Disclosure

The present disclosure provides methods of inducing or improving an anti-tumor response in a subject having a tumor comprising administering to the subject a priming dose of a vector expressing a Fas chimera protein, wherein the priming dose of the vector is administered to the subject prior to surgical removal of the tumor or a portion thereof. In particular aspects, the anti-tumor response is induced or improved after the administration compared to the anti-tumor response in a subject not receiving a priming dose of the vector prior to surgical removal of the tumor or a portion thereof or in a subject not administered the vector. In some aspects, the anti-tumor response is an anti-tumor immune response.

The present disclosure also provides methods for inducing or improving tumor infiltrating lymphocyte (TIL) density in a subject having a tumor comprising administering to the subject a priming dose of a vector expressing a Fas chimera protein, wherein the priming dose of the vector is administered to the subject prior to surgical removal of the tumor or a portion thereof. In particular aspects, the TIL density at the tumor site is induced or improved after the administration compared to the TIL density in a tumor of a subject not receiving a priming dose of the vector prior to surgical removal of the tumor or a portion thereof or in a subject not administered the vector.

The present disclosure also provides a method of inhibiting or reducing angiogenesis in a subject having a tumor comprising administering to the subject a priming dose of a vector expressing a Fas chimera protein, wherein the priming dose of the vector is administered prior to surgical removal of the tumor or a portion thereof. In particular aspects, the angiogenesis is inhibited or reduced after the administration compared to the angiogenesis in a subject not receiving a priming dose of the vector prior to surgical removal of the tumor or a portion thereof or in a subject not administered the vector.

Some aspects of the present disclosure are directed to a method of inducing apoptosis of an endothelial cell in a tumor of a subject in need thereof comprising administering to the subject a priming dose of a vector expressing a Fas chimera protein, wherein the priming dose of the vector is administered prior to surgical removal of the tumor or a portion thereof. In particular aspects, the apoptosis of endothelial cells in a tumor of the subject is augmented or enhanced compared to the apoptosis of endothelial cells in a tumor of a subject not receiving a priming dose of the vector prior to surgical removal of the tumor or a portion thereof or in a subject not administered the vector.

The present disclosure further provides a method of reducing or inhibiting the size of a tumor in a subject in need thereof comprising administering to the subject a priming dose of a vector expressing a Fas chimera protein, wherein the priming dose of the vector is administered prior to surgical removal of the tumor or a portion thereof. In particular aspects, the size of the tumor is reduced or inhibited after the administration compared to the size of the tumor in a subject not receiving a priming dose of the vector prior to surgical removal of the tumor or a portion thereof or in a subject not administered the vector.

The present disclosure also includes a method of treating a disease or condition associated with a tumor in a subject comprising administering to the subject a priming dose of a vector expressing a Fas chimera protein, wherein the priming dose of the vector is administered prior to surgical removal of the tumor or a portion thereof. In particular aspects, the disease or condition is treated after the administration compared to the disease or condition in a subject not receiving a priming dose of the vector prior to surgical removal of the tumor or a portion thereof or in a subject not administered the vector.

Tumor growth can be measured by techniques known in the art, including but not limited to magnetic resonance imaging (MM) scan, functional MM (fMRI) scan, computerized tomography (CT) scan, or positron emission tomography (PET) scan. In a particular aspect, the growth of the tumor is measured by MRI. In some aspects, the tumor of the subject is a recurrent tumor that arose during treatment with the vector. In yet other embodiments, the tumor of the subject is a metastatic tumor that arose during treatment with the vector.

The term “subject” or “individual” or “animal” or “patient” or “mammal,” is meant any subject, particularly a mammalian subject, having or being expected to have an anti-tumor response. In some aspects, the subject is a human. In some aspects, the subject is a cancer patient.

In one aspect, the subject is in need of improved or induced anti-tumor response. In a particular aspect, the subject in need of improved or induced anti-tumor response has a tumor or a metastasis thereof, wherein the methods of the disclosure treat, decrease, or reduce the size of a tumor or a metastasis thereof. In another aspect, the subject in need of improved or induced anti-tumor response needs angiogenesis inhibition, wherein the methods of the present disclosure treat, decrease, prevent, or reduce angiogenesis. In other aspects, the subject in need of improved or induced anti-tumor response has cancer, wherein the methods of the present disclosure treat cancer.

In one aspect, the subject is in need of reducing or inhibiting the size of a tumor in the subject. In a particular aspect, the subject in need of reducing or inhibiting the size of a tumor has a tumor or a metastasis thereof, wherein the methods of the disclosure treat, decrease, or reduce the size of a tumor or a metastasis thereof. In another aspect, the subject in need of reducing or inhibiting the size of a tumor needs angiogenesis inhibition, wherein the methods of the present disclosure treat, decrease, prevent, or reduce angiogenesis. In other aspects, the subject in need of reducing or inhibiting the size of a tumor has cancer, wherein the methods of the present disclosure treat cancer.

In one aspect, the subject is in need of inhibiting or reducing angiogenesis. In a particular aspect, the subject in need of inhibiting or reducing angiogenesis has a tumor or a metastasis thereof, wherein the methods of the disclosure treat, decrease, or reduce the size of a tumor or a metastasis thereof. In another aspect, the subject in need of inhibiting or reducing angiogenesis has a tumor or a metastasis thereof, wherein the methods of the present disclosure treat, decrease, prevent, or reduce angiogenesis. In other aspects, the subject in need of inhibiting or reducing angiogenesis has cancer, wherein the methods of the present disclosure treat cancer.

In one aspect, the subject is in need of inducing apoptosis in endothelial cells in a tumor. In a particular aspect, the subject in need of inducing apoptosis in endothelial cells in a tumor has a tumor or a metastasis thereof, wherein the methods of the disclosure treat, decrease, or reduce the size of a tumor or a metastasis thereof. In another aspect, the subject in need of inducing apoptosis in endothelial cells in a tumor needs angiogenesis inhibition, wherein the methods of the present disclosure treat, decrease, prevent, or reduce angiogenesis. In other aspects, the subject in need of inducing apoptosis in endothelial cells in a tumor has cancer, wherein the methods of the present disclosure treat cancer.

In one aspect, the subject is in need of treatment of a disease or condition associated with a tumor. In a particular aspect, the subject in need of treatment of a disease or condition associated with a tumor has a tumor or a metastasis thereof, wherein the methods of the disclosure treat, decrease, or reduce the size of a tumor or a metastasis thereof. In another aspect, the subject in need of treatment of a disease or condition associated with a tumor needs angiogenesis inhibition, wherein the methods of the present disclosure treat, decrease, prevent, or reduce angiogenesis. In other aspects, the subject in need of treatment of a disease or condition associated with a tumor has cancer, wherein the methods of the present disclosure treat cancer.

A. Priming Dose

In the methods of the present disclosure, the priming dose of the vector can be administered at different times prior to surgical removal of the tumor or a portion thereof. In some aspects, the priming dose of the vector is administered about 7 days, about 8 days, about 9 days, about 10 days, about 11 days, about 12 days, about 13 days, about 2 weeks, about 15 days, about 16 days, about 17 days, about 18 days, about 19 days, about 20 days, about 3 weeks, about 22 days, about 23 days, about 24 days, about 25 days, about 26 days, about 27 days, about 4 weeks, about a month, about 5 weeks, about 6 weeks, about 7 weeks, about 8 weeks, about 2 months, about 3 months, about 4 months, about 5 months, or about 6 months before surgical removal of the tumor or a portion thereof. In some aspects, the priming dose of the vector is administered about 9 days, about 10 days, about 11 days, about 12 days, about 13 days, about 14 days, about 15 days, about 16 days, about 17 days, about 18 days, or about 19 days prior to surgical removal of the tumor or a portion thereof. In some aspects, the priming dose of the vector is administered between about 9 days to about 19 days, between about 10 days to about 18 days, between about 11 days to about 17 days, between about 12 days to about 16 days, or between about 13 days to about 15 days prior to surgical removal of the tumor or a portion thereof. In a particular aspect, the priming dose of the vector is administered about 14 days prior to surgical removal of the tumor or a portion thereof.

The dose of the vector administered as part of the present disclosure can be measured in virus particles (VPs). An effective amount of the priming dose of the vector includes, but is not limited to equal to or less than about 1×10¹⁶, 1×10¹⁵, 1×10¹⁴, 5×10¹³, 4×10¹³, 3×10¹³, 2×10¹³, 1×10¹³, 9×10¹², 8×10¹², 7×10¹², 6×10¹², 5×10¹², 4×10¹², 3×10¹², 2×10¹², 1×10¹², 9×10¹¹, 8×10¹¹, 7×10¹¹, 6×10¹¹, 5×10¹¹, 4×10¹¹, 3×10¹¹, 2×10¹¹, 1×10¹¹, 9×10¹⁰, 8×10¹⁰, 7×10¹⁰, 6×10¹⁰, 5×10¹⁰, 4×10¹⁰, 3×10¹⁰, 2×10¹⁰, or 1×10¹⁰ virus particles. In other embodiments, an effective amount of the priming dose of the vector is about 1×10¹⁰ to about 1×10¹⁶, about 1×10¹¹ to about 1×10¹⁵, about 1×10¹¹ to about 1×10¹⁶, about 1×10¹² to about 1×10¹⁵, about 1×10¹² to about 1×10¹⁶, about 1×10¹² to about 1×10¹⁴, about 5×10¹² to about 1×10¹⁶, about 5×10¹² to about 1×10¹⁵, about 5×10¹² to about 1×10¹⁴, about 1×10¹² to about 1×10¹³, about 1×10¹³ to about 1×10¹⁴ virus particles.

In some aspects, the priming dose of the vector is administered at an effective amount of at least about 1×10¹¹ virus particles. In some aspects, the priming dose of the vector is administered at an effective amount of at least about 1×10¹² virus particles. In some aspects, the priming dose of the vector is administered at an effective amount of at least about 1×10¹³ virus particles. In some aspects, the priming dose of the vector is administered at an effective amount of at least about 3×10¹³ virus particles. In some aspects, the priming dose of the vector is administered at an effective amount of at least about 1×10¹⁴ virus particles. In some aspects, the priming dose of the vector is administered at an effective amount of at least about 1×10¹⁵ virus particles. In other embodiments, the priming dose of the vector is administered at an effective amount of at least about 1×10⁷, 1×10⁸, 1×10⁹, 1×10¹⁰, or 5×10¹⁰ virus particles.

B. Surgical Removal of Tumor or a Portion Thereof

In the present disclosure, surgical removal of a tumor or a portion thereof reduces tumor burden in the subject. Surgical removal can be a complete or partial resection of the tumor. In some aspects, surgical removal of the tumor is a partial resection of the tumor. In some aspects, surgical removal of the tumor removes about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, or about 99% of the tumor. In some aspects of the disclosure, surgical removal of the tumor is a complete resection of the tumor, wherein 100% of the tumor is removed.

C. Post-Surgical Dose

In some aspects of the present disclosure, the subject further receives a post-surgical dose of the vector after surgical removal of the tumor or a portion thereof. The post-surgical dose of the vector is administered after the subject recovers from surgical removal of the tumor or a portion thereof. In some aspects, the post-surgical dose of the vector is administered about 7 days, about 8 days, about 9 days, about 10 days, about 11 days, about 12 days, about 13 days, about 14 days, about 2 weeks, about 15 days, about 16 days, about 17 days, about 18 days, about 19 days, about 20 days, about 21 days, about 3 weeks, about 22 days, about 23 days, about 24 days, about 25 days, about 26 days, about 27 days, about 28 days, about 4 weeks, about 29 days, about 30 days, about a month, about 31 days, about 32 days, about 33 days, about 34 days, about 35 days, about 5 weeks, about 36 days, about 37 days, about 38 days, about 39 days, about 40 days, about 41 days, about 42 days, about 6 weeks, about 7 weeks, about 8 weeks, about 2 months, about 3 months, about 4 months, about 5 months, or about 6 months after surgical removal of the tumor or a portion thereof. In some aspects, the post-surgical dose of the vector is administered between about 14 days to about 35 days, between about 15 days to about 34 days, between about 16 days to about 34 days, between about 17 days to about 33 days, between about 18 days to about 32 days, between about 19 days to about 31 days, between about 20 days to about 30 days, between about 21 days to about 29 days, between about 22 days to about 28 days, between about 23 days to about 27 days, or between about 24 days to about 26 days after surgical removal of the tumor or a portion thereof. In a particular aspect, the post-surgical dose of the vector is administered not less than 14 days after surgery. In another aspect, the post-surgical dose of the vector is administered not more than 35 days after surgery. In another aspect, the post-surgical dose of the vector is administered not less than 14 days after surgery and not more than 35 days after surgery.

In some aspects, the post-surgical dose of the vector is repeatedly administered. In some aspects, the post-surgical dose of the vector is repeatedly administered every day, once in about 2 days, once in about 3 days, once in about 4 days, once in about 5 days, once in about 6 days, once in about 7 days, once in about 2 weeks, once in about 3 weeks, once in about 4 weeks, once in about 5 weeks, once in about 6 weeks, once in about 7 weeks, once in about 2 months, once in about 3 months, once in about 4 months, once in about 5 months, once in about 6 months, once in about 7 months, once in about 8 months, once in about 9 months, once in about 10 months, once in about 11 months, or once in about 12 months.

The dose of the vector administered as part of the present disclosure can be measured in virus particles (VPs). In one embodiment, the priming dose of the vector is lower than the post-surgical dose of the vector. In another embodiment, the priming dose of the vector is higher than the post-surgical dose of the vector. In another embodiment, the priming dose of the vector is the same as the post-surgical dose of the vector.

An effective amount of the post-surgical dose of the vector includes, but is not limited to equal to or less than about 1×10¹⁶, 1×10¹⁵, 1×10¹⁴, 5×10¹³, 4×10¹³, 3×10¹³, 2×10¹³, 1×10¹², 9×10¹², 8×10¹², 7×10¹², 6×10¹², 5×10¹², 4×10¹², 3×10¹², 2×10¹², 1×10¹², 9×10¹¹, 8×10¹¹, 7×10¹¹, 6×10¹¹, 5×10¹¹, 4×10¹¹, 3×10¹¹, 2×10¹¹, 1×10¹¹, 9×10¹⁰, 8×10¹⁰, 7×10¹⁰, 6×10¹⁰, 5×10¹⁰, 4×10¹⁰, 3×10¹⁰, 2×10¹⁰, or 1×10¹⁰ virus particles. In other embodiments, an effective amount of the priming dose of the vector is about 1×10¹⁰ to about 1×10¹⁶, about 1×10¹¹ to about 1×10¹⁵, about 1×10¹¹ to about 1×10¹⁶, about 1×10¹² to about 1×10¹⁵, about 1×10¹² to about 1×10¹⁶, about 1×10¹² to about 1×10¹⁴, about 5×10¹² to about 1×10¹⁶, about 5×10¹² to about 1×10¹⁵, about 5×10¹² to about 1×10¹⁴, about 1×10¹² to about 1×10¹³, about 1×10¹³ to about 1×10¹⁴ virus particles.

In some aspects, the post-surgical dose of the vector is administered at an effective amount of at least about 1×10¹¹ virus particles. In some aspects, the post-surgical dose of the vector is administered at an effective amount of at least about 1×10¹² virus particles. In some aspects, the post-surgical dose of the vector is administered at an effective amount of at least about 1×10¹³ virus particles. In some aspects, the post-surgical dose of the vector is administered at an effective amount of at least about 1×10¹⁴ virus particles. In some aspects, the post-surgical dose of the vector is administered at an effective amount of at least about 1×10¹⁵ virus particles. In other embodiments, the post-surgical dose of the vector is administered at an effective amount of at least about 1×10⁷, 1×10⁸, 1×10⁹, 1×10¹⁰, or 5×10¹⁰ virus particles.

D. Diseases and Conditions

The methods of the present disclosure are useful for stabilizing a disease or disorder associated with cancer. In some embodiments, with the cancer is metastatic colorectal cancer (mCRC), advanced nonsquamous non-small cell lung cancer (NSCLC), metastatic renal cell carcinoma (mRCC), glioblastoma multiforme (GBM), Müllerian cancer, ovarian cancer, peritoneal cancer, fallopian tube cancer, or uterine papillary serousspects. In some aspects, the present disclosure reduces the volume of malignant peritoneal fluid, e.g., ascites, reduces pain to the subject, prolongs survival of the subject, or any combinations thereof. The tumor that can be reduced, inhibited, or treated with the present disclosure can be a solid tumor, a primary tumor, or a metastatic tumor. The term “metastatic” or “metastasis” refers to tumor cells that are able to establish secondary tumor lesions in another parts or organ.

In the methods of the present disclosure, a “solid tumor” includes, but is not limited to, sarcoma, melanoma, carcinoma, or other solid tumor cancer. “Sarcoma” refers to a tumor which is made up of a substance like the embryonic connective tissue and is generally composed of closely packed cells embedded in a fibrillar or homogeneous substance. Sarcomas include, but are not limited to, chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” refers to a tumor arising from the melanocytic system of the skin and other organs. Melanomas include, for example, acra-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, metastatic melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas include, for example, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniform carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidernoid carcinoma, carcinoma mucosum, mucous carcinoma, carcinoma myxomatodes, naspharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma viflosum.

Additional cancers that may be inhibited or treated include, for example, Leukemia, Hodgkin's Disease, Non-Hodgkin's Lymphoma, multiple myeloma, neuroblastoma, breast cancer, ovarian cancer, lung cancer, (including non-small cell lung cancer (NSCLC)), rhabdomyosarcoma, primary thrombocytosis, primary macroglobulinemia, small-cell lung tumors, primary brain tumors, gliomas (including glioblastoma multiforme (GBM) and recurrent GBM), stomach cancer, colon cancer (including metastatic colorectal cancer (mCRC)), hepatobiliary cancer, malignant pancreatic insulanoma, malignant carcinoid, urinary bladder cancer, premalignant skin lesions, testicular cancer, lymphomas, thyroid cancer, papillary thyroid cancer, neuroblastoma, neuroendocrine cancer, esophageal cancer, genitourinary tract cancer, malignant hypercalcemia, cervical cancer, endometrial cancer, adrenal cortical cancer, prostate cancer, Müllerian cancer, ovarian cancer, peritoneal cancer, fallopian tube cancer, or uterine papillary serous carcinoma.

In a particular aspect, the tumor is a glioma. The term “glioma” refers to a tumor arising from glial cells of the brain or spine. Gliomas can include but are not limited to ependymomas, astrocytomas (including glioblastoma multiforme), oligodendrogliomas, brainstem gliomas, optic nerve gliomas, and mixed gliomas such as oligoastrocytomas. In a particular aspect, the tumor is associated with or derived from glioblastoma multiforme. In some aspects, the tumor is a recurrent tumor. In a particular aspect, the tumor is a recurrent glioblastoma multiforme.

### III. Nucleic Acid Constructs Comprising a Fas-chimera Gene and an Endothelial Cell Specific Promoter

The present disclosure provides methods of anti-tumor therapy comprising administering to a subject having a tumor a priming dose of a vector which comprises a Fas-chimera gene operably linked to an endothelial cell specific promoter, wherein the vector is administered prior to surgical removal of the tumor or a portion thereof. The present disclosure further provides methods of administering to the subject one or more post-surgical doses of the Fas-chimera vector.

The gene encoding the Fas-chimera protein (or gene product), in the present disclosure can be linked to an endothelial cell-specific promoter, which directs expression of the Fas-chimera gene product in an endothelial cell. Expression of such a cytotoxic gene product is useful in a situation where excessive neo-vascularization or blood vessel growth is not desirable, e.g., in a tumor.

A. Fas-Chimera

A Fas-chimera protein expressed by the nucleic acid construct of the invention comprises at least two “death receptor” polypeptides, each of the polypeptides is derived from a different protein. The first polypeptide of the Fas-chimera protein comprises a ligand binding domain of Tumor Necrosis Factor Receptor 1 (TNFR1). The second polypeptide of the Fas-chimera protein comprises an effector domain of a Fas polypeptide.

The ligand binding domain of TNFR1 can be any domain that binds to a TNFR1 ligand. In one embodiment, the TNFR1 ligand is TNF-α. In another embodiment, the TNFR1 ligand is lymphotoxin-α. The ligand binding domain of TNFR1 can be an extracellular domain of TNFR1 or any fragments, variants, derivatives, or analogues thereof. Non-limiting examples of the TNFR1 ligand binding domain are described below.

The effector domain of a Fas polypeptide useful for the invention comprises any Fas domains that form death-inducing signaling complex (DISC), thereby inducing apoptosis. In one embodiment, an effector domain of a Fas polypeptide comprises an intracellular domain, a trans-membrane domain, or both. Non-limiting examples of Fas polypeptide effector domains are described below.

The TNFR1 and the Fas polypeptide can be linked by a peptide bond or by a linker. The linker connecting the TNFR1 ligand binding domain with the Fas effector domain can be a polypeptide linker or a non-peptide linker. For example, a linker for the Fas-chimera protein can comprise one or more glycine, serine, leucine, or any combinations thereof. In one embodiment, a linker useful for the invention comprises Ser-Leu. In another embodiment, a linker useful for the invention comprises (GGGS)n, (Denise et al. J. Biol. Chem. 277:35035-35043 (2002)), wherein n can be 0, 1, 2, 3, 4, 5, 6, 7, 8, 9, or 10 or more (SEQ ID NO: 27).

1. Tumor Necrosis Factor Receptor 1

The full-length human TNFR1 polypeptide is 455 amino acids in length and is also known as TNF-R1, Tumor necrosis factor receptor type I (TNFRI), TNFR-I, TNFRSF1A, TNFAR, p55, P60, or CD120a. Naturally-occurring human TNFR1 polypeptide is known to bind to TNF-α or homotrimeric lymphotoxin-α. Binding of TNF-α to the extracellular domain leads to homotrimerization of TNFR1, which then interacts specifically with the death domain of Tumor Necrosis Factor Receptor Type 1-Associated Death Domain Protein (TRADD). Various TRADD-interacting proteins such as TNF Receptor Associated Factors (TRAFS), Receptor-Interacting Serine/Threonine-Protein Kinase 1 (RIPK1), and Fas-Associated Protein with Death Domain (FADD) are recruited to the complex by their association with TRADD. The complex activates at least two distinct signaling cascades, apoptosis and NF-kappa-B signaling.

A 455 aa polypeptide sequence reported as a human TNFR1 polypeptide sequence has the identifier number P19438-1 in the UniProtKB database. This human TNFR1 polypeptide sequence is designated herein as isoform A and SEQ ID NO: 2. SEQ ID NO: 1 is a nucleotide sequence encoding SEQ ID NO: 2. A polypeptide sequence of 108 aa was reported as an isoform of the human TNFR1 polypeptide sequence and has the identifier number P19438-2 in the UniProtKB database. The 108 aa polypeptide corresponds to amino acids 1 to 108 of isoform A (SEQ ID NO: 2) and is designated herein as isoform B. Another variant of the human TNFR1 polypeptide having 232 aa was reported as the identifier number P19438-3 in the UniProtKB database. The 232 aa polypeptide corresponds to amino acids 1 to 232 of isoform A (SEQ ID NO: 2) and is designated herein as isoform C. Additional natural variants of human TNFR1 include, but are not limited to, the TNFR1 polypeptide of isoforms A, B, and C comprising one or more mutations selected from the group consisting of H51Q, C59R, C59S, C62G, C62Y, P75L, T79M, C81F, C99S, S115G, C117R, C117Y, R121P, R121Q, P305T, and any combinations thereof. Other known TNFR1 variants include the TNFR1 polypeptide of isoforms A, B, and C comprising L13LILPQ, K255E, S286G, R394L, 412:Missing, GPAA443-446APP, or any combinations thereof.

Table 1 shows the human wild-type TNFR1 amino acid sequence and a nucleotide sequence encoding the wild-type TNFR1.

TABLE 1
TNFR1 Sequences

| SEQ ID | |
|---|---|
| No. | Sequences |
| Amino | MGLSTVPDLLLPLVLLELLVGIYPSGVIGLVPHLGDREKRDSVCPQGKYIHPQNNSICCT |
| acid | KCHKGTYLYNDCPGPGQDTDCRECESGSFTASENHLRHCLSCSKCRKEMGQVEISSCTVD |
| sequence | RDTVCGCRKNQYRHYWSENLFQCFNCSLCLNGTVHLSCQEKQNTVCTCHAGFFLRENECV |
| of TNFR1 | SCSNCKKSLECTKLCLPQIENVKGTEDSGTTVLLPLVIFFGLCLLSLLFIGLMYRYQRWK |
| (SEQ ID | SKLYSIVCGKSTPEKEGELEGTTTKPLAPNPSFSPTPGFTPTLGFSPVPSSTFTSSSTYT |
| NO: 2) | PGDCPNFAAPRREVAPPYQGADPILATALASDPIPNPLQKWEDSAHKPQSLDTDDPATLY |
| | AVVENVPPLRWKEFVRRLGLSDHEIDRLELQNGRCLREAQYSMLATWRRRTPRREATLEL |
| | LGRVLRDMDLLGCLEDIEEALCGPAALPPAPSLLR |
| Nucleotide | Atgggcctctccaccgtgcctgacctgctgctgccgctggtgctcctggagctgttggtg |
| Sequence | Ggaatatacccctcaggggttattggactggtccctcacctaggggacagggagaagaga |
| encoding | Gatagtgtgtgtccccaaggaaaatatatccaccctcaaaataattcgatttgctgtacc |
| TNFR1 | Aagtgccacaaaggaacctacttgtacaatgactgtccaggcccggggcaggatacggac |
| (SEQ ID | Tgcagggagtgtgagagcggctccttcaccgcttcagaaaaccacctcagacactgcctc |
| NO: 1) | Agctgctccaaatgccgaaaggaaatgggtcaggtggagatctcttcttgcacagtggac |
| | Cgggacaccgtgtgtggctgcaggaagaaccagtaccggcattattggagtgaaaacctt |
| | Ttccagtgcttcaattgcagcctctgcctcaatgggaccgtgcacctctcctgccaggag |
| | Aaacagaacaccgtgtgcacctgccatgcaggtttctttctaagagaaaacgagtgtgtc |
| | Tcctgtagtaactgtaagaaaagcctggagtgcacgaagttgtgcctaccccagattgag |
| | Aatgttaagggcactgaggactcaggcaccacagtgctgttgcccctggtcattttcttt |
| | Ggtctttgccttttatccctcctcttcattggtttaatgtatcgctaccaacggtggaag |
| | Tccaagctctactccattgtttgtgggaaatcgacacctgaaaaagagggggagcttgaa |
| | Ggaactactactaagcccctggccccaaacccaagcttcagtcccactccaggcttcacc |
| | Cccaccctgggcttcagtcccgtgcccagttccaccttcacctccagctccacctatacc |
| | Cccggtgactgtcccaactttgcggctccccgcagagaggtggcaccaccctatcagggg |
| | Gctgaccccatccttgcgacagccctcgcctccgaccccatccccaacccccttcagaag |
| | Tgggaggacagcgcccacaagccacagagcctagacactgatgaccccgcgacgctgtac |
| | Gccgtggtggagaacgtgcccccgttgcgctggaaggaattcgtgcggcgcctagggctg |
| | Agcgaccacgagatcgatcggctggagctgcagaacgggcgctgcctgcgcgaggcgcaa |
| | Tacagcatgctggcgacctggaggcggcgcacgccgcggcgcgaggccacgctggagctg |
| | Ctgggacgcgtgctccgcgacatggacctgctgggctgcctggaggacatcgaggaggcg |
| | ctttgcggccccgccgccctcccgcccgcgcccagtcttctcaga |
| Amino | MGLSTVPDLLLPLVLLELLVGIYPSGVIGLVPHLGDREKRDSVCPQGKYIHPQNNSICCT |
| acid | KCHKGTYLYNDCPGPGQDTDCRECESGSFTASENHLRHCLSCSKCRKEMGQVEISSCTVD |
| sequence | RDTVCGCRKNQYRHYWSENLFQCFNCSLCLNGTVHLSCQEKQNTVCTCHAGFFLRENECV |
| of a | SCSNCKKSLECTKLCLP |
| Ligand | |
| Binding | |
| Domain of | |
| TNFR1 | |
| (SEQ ID | |
| NO: 4) | |
| Nucleotide | atgggcctct ccaccgtgcc tgacctgctg ctgccgctgg tgctcctgga |
| sequence | gctgttggtg ggaatatacc cctcaggggt tattggactg gtccctcacc |
| encoding a | taggggacag ggagaagaga gatagtgtgt gtccccaagg aaaatatatc |
| Ligand | caccctcaaa ataattcgat ttgctgtacc aagtgccaca aaggaaccta |
| Binding | cttgtacaat gactgtccag gcccggggca ggatacggac tgcagggagt |
| Domain of | gtgagagcgg ctccttcacc gcttcagaaa accacctcag acactgcctc |
| TNFR1 | agctgctcca aatgccgaaa ggaaatgggt caggtggaga tctcttcttg |
| (SEQ ID | cacagtggac cgggacaccg tgtgtggctg caggaagaac cagtaccggc |
| NO: 3) | attattggag tgaaaacctt ttccagtgct tcaattgcag cctctgcctc |
| | aatgggaccg tgcacctctc ctgccaggag aaacagaaca ccgtgtgcac |
| | ctgccatgca ggtttctttc taagagaaaa cgagtgtgtc tcctgtagta |
| | actgtaagaa aagcctggag tgcacgaagt tgtgcctacc a |

The mouse TNFR1 polypeptide sequence and its variants are also reported. The 454 aa mouse TNFR1 polypeptide has the identifier number P25118 in UniProtKB database. TNFR1 polypeptides known in other animals include, but are not limited to, rat (e.g., P22934 in the UniProtKB database), cow (e.g., 019131 in the UniProtKB database), pig (e.g., P50555 in the UniProtKB database), or horse (e.g., D1MH71 in the UniProtKB database).

The full-length TNFR1 can be cleaved into two chains, (1) TNF Receptor Superfamily Member 1A, membrane form (i.e., amino acids 22 to 455 corresponding to full-length TNFR1) and (2) TNF-binding protein 1 (TBPI) (i.e., amino acids 41 to 291 corresponding to full-length TNFR1). The full-length human TNFR1 polypeptide consists of a signal sequence (amino acids 1 to 21 of SEQ ID NO: 2), an extracellular domain (amino acids 22 to 211 of SEQ ID NO: 2), a trans-membrane domain (amino acids 212 to 234 of SEQ ID NO: 2), and a cytoplasmic domain (amino acids 235 to 455 of SEQ ID NO: 2). The TNFR1 extracellular domain comprises four cysteine repeat regions, TNFR-Cys1 (amino acids 43 to 82 corresponding to SEQ ID NO: 2), TNFR-Cys2 (amino acids 83 to 125 corresponding to SEQ ID NO: 2), TNFR-Cys3 (amino acids 126 to 166 corresponding to SEQ ID NO: 2), and TNFR-Cys4 (amino acids 167 to 196 corresponding to SEQ ID NO: 2).

As one of skill in the art will appreciate, the beginning and ending residues of the domains listed above can vary depending upon the computer modeling program used or the method used for determining the domain. As such, various functional domains of TNFR1 may vary from those defined above.

In one embodiment, a ligand binding domain of TNFR1 useful for the Fas-chimera protein comprises, consists essentially of, or consists of an extracellular domain of TNFR1, or any fragment, variant, derivative, or analogue thereof, wherein the extracellular domain of TNFR1, or any fragment, variant, derivative, or analogue thereof binds to TNF-α. In another embodiment, a ligand binding domain of TNFR1 comprises TNFR-Cys1; TNFR-Cys2; TNFR-Cys3; TNFR-Cys4; TNFR-Cys1 and TNFR-Cys2; TNFR-Cys1 and TNFR-Cys3; TNFR-Cys1 and TNFR-Cys4; TNFR-Cys2 and TNFR-Cys3; TNFR-Cys2 and TNFR-Cys4; TNFR-Cys3 and TNFR-Cys4; TNFR-Cys1, TNFR-Cys2, and TNFR-Cys3; TNFR-Cys1, TNFR-Cys2, and TNFR-Cys4; TNFR-Cys2, TNFR-Cys3, and TNFR-Cys4; or TNFR-Cys1, TNFR-Cys2, TNFR-Cys3, and TNFR-Cys4. In other embodiments, a ligand binding domain of TNFR1 in the Fas-chimera protein comprises TNF binding protein I. In yet other embodiments, a TNFR1 ligand binding domain of the Fas-chimera protein comprises, consists essentially of, or consists of an amino acid sequence at least 60%, 70%, 80%, 90%, 95%, 96%, 97%, 98%, 99%, or 100% identical to amino acids 22 to 190, amino acids 22 to 191, amino acids 22 to 192, amino acids 22 to 193, amino acids 22 to 194, amino acids 22 to 195, amino acids 22 to 196, amino acids 22 to 197, amino acids 22 to 198, amino acids 22 to 199, amino acids 22 to 200, amino acids 22 to 201, amino acids 22 to 202, amino acids 22 to 203, amino acids 22 to 204, amino acids 22 to 205, amino acids 22 to 206, amino acids 22 to 207, amino acids 22 to 208, amino acids 22 to 209, amino acids 22 to 210, or amino acids 22 to 211 of SEQ ID NO: 2, wherein the ligand binding domain binds to a TNFR1 ligand, e.g., TNF-α.

In other embodiments, the ligand binding domain of TNFR1 further comprises a signal peptide. One example of the suitable signal peptides is the signal peptide of TNFR1, e.g., amino acids 1 to 21 of SEQ ID NO: 2. In yet other embodiments, a ligand binding domain of the Fas-chimera gene product comprises, consists essentially of, or consists of an amino acid sequence at least 60%, 70%, 80%, 90%, 95%, 96%, 97%, 98%, 99%, or 100% identical to amino acids 1 to 190, amino acids 1 to 191, amino acids 1 to 192, amino acids 1 to 193, amino acids 1 to 194, amino acids 1 to 195, amino acids 1 to 196, amino acids 1 to 197, amino acids 1 to 198, amino acids 1 to 199, amino acids 1 to 200, amino acids 1 to 201, amino acids 1 to 202, amino acids 1 to 203, amino acids 1 to 204, amino acids 1 to 205, amino acids 1 to 206, amino acids 1 to 207, amino acids 1 to 208, amino acids 1 to 209, amino acids 1 to 210, or amino acids 1 to 211 of SEQ ID NO: 2, wherein the ligand binding domain binds to a TNFR1 ligand, e.g., TNF-α. In a specific embodiment, a TNFR1 ligand binding domain of the Fas-chimera protein comprises, consists essentially of, or consists of an amino acid sequence at least 60%, 70%, 80%, 90%, 95%, 96%, 97%, 98%, 99%, or 100% identical to SEQ ID NO: 4, wherein the ligand binding domain binds to a TNFR1 ligand, e.g., TNF-α.

In yet other embodiments, the ligand binding domain of TNFR1 is encoded by a nucleotide sequence at least 60%, 70%, 80%, 90%, 95%, 96%, 97%, 98%, 99%, or 100% identical to SEQ ID NO: 3.

In still other embodiments, a TNFR1 ligand binding domain of the Fas-chimera protein comprises, consists essentially of, or consists of an amino acid sequence at least 60%, 70%, 80%, 90%, 95%, 96%, 97%, 98%, 99%, or 100% identical to amino acids 22 to 108 of SEQ ID NO: 2 (TNFR1 isoform B), amino acids 22 to 232 of SEQ ID NO: 2 (TNFR1 isoform C), or amino acids 44 to 291 of SEQ ID NO: 2 (TBP1), wherein the ligand binding domain binds to a TNFR1 ligand, e.g., TNF-α.

2. Fas Polypeptide

The full-length human Fas polypeptide is 335 amino acids in length and is also known as Tumor Necrosis Factor Receptor Superfamily Member 6, Apo-1 antigen, Apoptosis-mediating surface antigen Fas, Fas Ligand (FasL) receptor, or CD95. Naturally occurring Fas polypeptide is a receptor for TNFSF6/FasL. When the Fas polypeptide binds to the Fas ligand (FasL), the interaction between Fas and FasL results in the formation of the death-inducing signaling complex (DISC), which contains the FADD, caspase-8 and caspase-10. In some types of cells (type I), processed caspase-8 directly activates other members of the caspase family, and triggers the execution of apoptosis of the cell. In other types of cells (type II), the Fas-DISC starts a feedback loop that spirals into increasing release of proapoptotic factors from mitochondria and the amplified activation of caspase-8. Fas-mediated apoptosis may have a role in the induction of peripheral tolerance, in the antigen-stimulated suicide of mature cells or both.

A 335 aa polypeptide sequence reported as a human Fas polypeptide sequence has the identifier number P25445-1 in the UniProtKB database. This human Fas polypeptide sequence is designated herein as SEQ ID NO: 6. SEQ ID NO: 5 is a nucleotide sequence encoding SEQ ID NO: 6. The nucleotide sequence encoding the Fas polypeptide is also known as APT1, FAS1, or TNFRSF6. The full-length Fas polypeptide contains a signal peptide (amino acids 1 to 25 corresponding to SEQ ID NO: 6), an extracellular domain (amino acids 26 to 173 corresponding to SEQ ID NO: 6), a trans-membrane domain (amino acids 174 to 190 corresponding to SEQ ID NO: 6), and an intracellular (or cytoplasmic) domain (amino acids 191 to 335 corresponding to SEQ ID NO: 6). The intracellular domain contains a death domain (e.g., amino acids 230 to 314 corresponding to SEQ ID NO: 6).

As one of skill in the art will appreciate, the beginning and ending residues of the domains listed above may vary depending upon the computer modeling program used or the method used for determining the domain. As such, various functional domains of Fas may vary from those defined above. Table 2 shows the wild-type human Fasamino acid sequence and a nucleotide sequence encoding the Fas protein.

TABLE 2
Fas Sequences

| | Sequences |
|---|---|
| Amino acid | MLGIWTLLPLVLTSVARLSSKSVNAQVTDINSKGLELRKTVTTVETQNLEGLHHDGQFCH |
| sequence of | KPCPPGERKARDCTVNGDEPDCVPCQEGKEYTDKAHFSSKCRRCRLCDEGHGLEVEINCT |
| human Fas | RTQNTKCRCKPNFFCNSTVCEHCDPCTKCEHGIIKECTLTSNTKCKEEGSRSNLGWLCLL |
| protein | LLPIPLIVWVKRKEVQKTCRKHRKENQGSHESPTLNPETVAINLSDVDLSKYITTIAGVM |
| (SEQ ID | TLSQVKGFVRKNGVNEAKIDEIKNDNVQDTAEQKVQLLRNWHQLHGKKEAYDTLIKDLKK |
| NO: 6) | ANLCTLAEKIQTIILKDITSDSENSNFRNEIQSLV |
| Nucleotide | Atgctgggcatctggaccctcctacctctggttcttacgtctgttgctagattatcgtcc |
| sequence | Aaaagtgttaatgcccaagtgactgacatcaactccaagggattggaattgaggaagact |
| encoding | Gttactacagttgagactcagaacttggaaggcctgcatcatgatggccaattctgccat |
| human Fas | Aagccctgtcctccaggtgaaaggaaagctagggactgcacagtcaatggggatgaacca |
| sequence | Gactgcgtgccctgccaagaagggaaggagtacacagacaaagcccatttttcttccaaa |
| (SEQ ID | Tgcagaagatgtagattgtgtgatgaaggacatggcttagaagtggaaataaactgcacc |
| NO 5) | Cggacccagaataccaagtgcagatgtaaaccaaactttttttgtaactctactgtatgt |
| | Gaacactgtgacccttgcaccaaatgtgaacatggaatcatcaaggaatgcacactcacc |
| | Agcaacaccaagtgcaaagaggaaggatccagatctaacttggggtggctttgtcttctt |
| | Cttttgccaattccactaattgtttgggtgaagagaaaggaagtacagaaaacatgcaga |
| | Aagcacagaaaggaaaaccaaggttctcatgaatctccaactttaaatcctgaaacagtg |
| | Gcaataaatttatctgatgttgacttgagtaaatatatcaccactattgctggagtcatg |
| | Acactaagtcaagttaaaggctttgttcgaaagaatggtgtcaatgaagccaaaatagat |
| | Gagatcaagaatgacaatgtccaagacacagcagaacagaaagttcaactgcttcgtaat |
| | Tggcatcaacttcatggaaagaaagaagcgtatgacacattgattaaagatctcaaaaaa |
| | Gccaatctttgtactcttgcagagaaaattcagactatcatcctcaaggacattactagt |
| | Gactcagaaaattcaaacttcagaaatgaaatccaaagcttggtctag |
| Amino acid | GSRSNLGWLCLLLLPIPLIVWVKRKEVQKTCRKHRKENQGS |
| sequence of | HESPTLNPETVAINLSDVDLSKYITTIAGVMTLSQVKGFVR |
| an Effector | KNGVNEAKIDEIKNDNVQDTAEQKVQLLRNWHQLHGKKEAY |
| Domain of | DTLIKDLKKANLCTLAEKIQTIILKDITSDSENSNFRNEIQ |
| Fas (SEQ ID | SLV |
| NO: 8) | |
| Nucleotide | Aggatccagatctaacttggggtggctttgtcttcttcttttgccaattccactaatt |
| sequence | Gtttgggtgaagagaaaggaagtacagaaaacatgcagaaagcacagaaaggaaaacc |
| encoding an | Aaggttctcatgaatctccaaccttaaatcctgaaacagtggcaataaatttatctga |
| Effector | Tgttgacttgagtaaatatatcaccactattgctggagtcatgacactaagtcaagtt |
| Domain of | Aaaggctttgttcgaaagaatggtgtcaatgaagccaaaatagatgagatcaagaatg |
| Fas (SEQ ID | Acaatgtccaagacacagcagaacagaaagttcaactgcttcgtaattggcatcaact |
| NO: 7) | Tcatggaaagaaagaagcgtatgacacattgattaaagatctcaaaaaagccaatctt |
| | Tgtactcttgcagagaaaattcagactatcatcctcaaggacattactagtgactcag |
| | aaaattcaaacttcagaaatgaaatccaaagcttggtctag |

The mouse Fas polypeptide sequence and its variants are also reported. The 327 aa mouse Fas polypeptide has the identifier number P25446 in UniProtKB database. Fas polypeptides known in other animals include, but are not limited to, Old World monkey (e.g., Q9BDN4 in the UniProtKB database), Rhesus monkey (e.g., Q9BDP2 in the UniProtKB database), rat (e.g., Q63199 in the UniProtKB database), or cow (e.g., P51867 in the UniProtKB database).

Based on the sequence variation in the Fas polypeptide, a person of ordinary skill in the art can identify sequence variations in the effector domain of the Fas polypeptide. For example, natural variants of the Fas effector domains can include one or more substitutions or mutations of C178R, L180F, P183L, I184V, T1981, Y232C, T241K, T241P, V249L, R250P, R250Q, G253D, G253S, N255D, A257D, I259R, D260G, D260V, D260Y, I262S, N264K, T270I, T270K, E272G, E272K, L278F, K299N, T3051, 1310S, or any combinations thereof.

In one embodiment, an effector domain of the Fas polypeptide useful for the invention comprises a death domain of the Fas polypeptide. In another embodiment, an effector domain of the Faspolypeptide comprises, consists essentially of, or consists of an amino acid sequence at least 60%, 70%, 80%, 90%, 95%, 96%, 97%, 98%, 99%, or 100% identical to amino acids 230 to 314 of SEQ ID NO: 6. In other embodiments, an effector domain of the Fas polypeptide comprises an intracellular domain of the Fas polypeptide. In yet other embodiments, an effector domain of the Fas polypeptide comprises an amino acid sequence at least 60%, 70%, 80%, 90%, 95%, 96%, 97%, 98%, 99%, or 100% identical to amino acids 185 to 335, amino acids 186 to 335, amino acids 187 to 335, amino acids 188 to 335, amino acids 189 to 335, amino acids 190 to 335, amino acids 191 to 335, amino acids 192 to 335, amino acids 193 to 335, amino acids 194 to 335, amino acids 195 to 335, amino acids 196 to 335, amino acids 197 to 335, amino acids 198 to 335, or amino acids 199 to 335 of SEQ ID NO: 6.

In still other embodiments, the effector domain of the Fas polypeptide further comprises a trans-membrane domain of the Fas polypeptide. In yet other embodiments, an effector domain of the Fas polypeptide comprises an amino acid sequence at least about 60%, 70%, 80%, 90%, 95%, 96%, 97%, 98%, 99%, or 100% identical to amino acids 174 to 335 of SEQ ID NO: 6. In some embodiments, an effector domain of the Fas polypeptide further comprises about ten, about nine, about eight, about seven, about six, about five, about four, about three, about two, or about one amino acid from the C-terminal portion of the Fas extracellular domain. In certain embodiments, an effector domain of the Fas polypeptide comprises an amino acid sequence at least 60%, 70%, 80%, 90%, 95%, 96%, 97%, 98%, 99%, or 100% identical to amino acids 179 to 335, amino acids 178 to 335, amino acids 177 to 335, amino acids 176 to 335, amino acids 175 to 335, amino acids 174 to 335, amino acids 173 to 335, amino acids 172 to 335, amino acids 171 to 335, amino acids 170 to 335, amino acids 169 to 335, amino acids 168 to 335, amino acids 167 to 335, amino acids 166 to 335, amino acids 165 to 335, amino acids 164 to 335, or amino acids 163 to 335 of SEQ ID NO: 6, wherein the effector domain forms a death-inducing signaling complex (DISC), activates caspase 8, or induces apoptosis.

In some embodiments, an effector domain of the Fas polypeptide comprises, consists essentially of, or consists of an amino acid sequence at least 70%, 80%, 90%, 95%, 96%, 97%, 98%, 99%, or 100% identical to SEQ ID NO: 8, wherein the effector domain forms a death-inducing signaling complex (DISC), activates caspase 8, or induces apoptosis.

In other embodiments, an effector domain of the Fas polypeptide is encoded by a nucleotide sequence at least 60%, 70%, 80%, 90%, 95%, 96%, 97%, 98%, 99%, or 100% identical to SEQ ID NO: 7.

In one embodiment, the Fas-chimera gene product for the invention comprises, consists essentially of, or consists of an amino acid sequence at least 70%, 80%, 90%, 95%, 96%, 97%, 98%, 99%, or 100% identical to SEQ ID NO: 10, wherein the Fas-chimera gene product induces apoptosis. In another embodiment, the Fas-chimera gene product is encoded by a nucleotide sequence at least 60%, 70%, 80%, 90%, 95%, 96%, 97%, 98%, 99%, or 100% identical to SEQ ID NO: 9, wherein the Fas-chimera gene product induces apoptosis.

B. Endothelial Cell-Specific Promoter

The nucleic acid construct comprising a Fas-chimera gene further comprises one or more expression control elements useful for regulating the expression of an operably linked Fas-chimera gene. The expression control elements include, but are not limited to, promoters, secretion signals, and other regulatory elements.

The nucleic acid construct useful for the present disclosure utilizes an endothelial cell-specific promoter to direct expression of the Fas-chimera protein in an endothelial cell, thereby inducing apoptosis of the endothelial cell.

For the purpose of the present disclosure, an endothelial cell-specific promoter can contain one or more cis-regulatory elements, which improve the endothelial cell-specificity of the promoters compared to the promoter without the cis-regulatory elements. In one example, the cis-regulatory element comprises a polynucleotide sequence that further promotes endothelial cell-specific transcription. In another aspect, the cis-regulatory element comprises a hypoxia response element. In other examples, the cis-regulatory element comprises both the polynucleotide sequence and a hypoxia response element.

In one embodiment, a cis-regulatory element useful for the invention comprises a nucleotide sequence at least 60%, 70%, 80%, 90%, 95%, 96%, 97%, 98%, 99%, or 100% identical to SEQ ID NO: 11 or SEQ ID NO: 12 (the complementary sequence of SEQ ID NO: 11), wherein the cis-regulatory element improves endothelial cell specificity of a promoter compared to a promoter without the cis-regulatory element. The cis-regulatory element can further comprise an additional nucleotide sequence at least 60%, 70%, 80%, 90%, 95%, 96%, 97%, 98%, 99%, or 100% identical to SEQ ID NO: 13 or SEQ ID NO: 14 (the complementary sequence of SEQ ID NO: 13).

In another embodiment, a cis-regulatory element for the invention comprises a nucleotide sequence at least 60%, 70%, 80%, 90%, 95%, 96%, 97%, 98%, 99%, or 100% identical to SEQ ID NO: 13 or SEQ ID NO: 14 (the complementary sequence of SEQ ID NO: 13), wherein the cis-regulatory element improves endothelial cell specificity of a promoter compared to a promoter without the cis-regulatory element. The cis-regulatory element can further comprise an additional nucleotide sequence at least 60%, 70%, 80%, 90%, 95%, 96%, 97%, 98%, 99%, or 100% identical to SEQ ID NO: 11 or SEQ ID NO: 12 (the complementary sequence of SEQ ID NO: 11).

In other embodiments, a cis-regulatory element for the invention comprises a nucleotide sequence at least 60%, 70%, 80%, 90%, 95%, 96%, 97%, 98%, 99%, or 100% identical to SEQ ID NO: 15 or SEQ ID NO: 16 (the complementary sequence of SEQ ID NO: 15), wherein the cis-regulatory element improves endothelial cell specificity of a promoter compared to a promoter without the cis-regulatory element. In yet other embodiments, a cis-regulatory element for the nucleic acid construct comprises SEQ ID NO: 7 or any fragments, variants, derivatives, or analogs thereof, wherein the fragments, variants, derivatives, or analogs improve endothelial cell specificity of a promoter compared to a promoter without the cis-regulatory element.

In some embodiments, a cis-regulatory element for the invention comprises a nucleotide sequence at least 60%, 70%, 80%, 90%, 95%, 96%, 97%, 98%, 99%, or 100% identical to SEQ ID NO: 22 or SEQ ID NO: 23, wherein the cis-regulatory element improves endothelial cell specificity of a promoter compared to a promoter without the cis-regulatory element. In yet other embodiments, a cis-regulatory element for the nucleic acid construct comprises SEQ ID NO: 22 or SEQ ID NO: 23 or any fragments, variants, derivatives, or analogs thereof, wherein the fragments, variants, derivatives, or analogs improve endothelial cell specificity of a promoter compared to a promoter without the cis-regulatory element.

In other embodiments, a cis-regulatory element for the invention comprises a nucleotide sequence at least 60%, 70%, 80%, 90%, 95%, 96%, 97%, 98%, 99%, or 100% identical to SEQ ID NO: 24 or SEQ ID NO: 25, wherein the cis-regulatory element improves endothelial cell specificity of a promoter compared to a promoter without the cis-regulatory element. In yet other embodiments, a cis-regulatory element for the nucleic acid construct comprises SEQ ID NO: 24 or SEQ ID NO: 25 or any fragments, variants, derivatives, or analogs thereof, wherein the fragments, variants, derivatives, or analogs improve endothelial cell specificity of a promoter compared to a promoter without the cis-regulatory element.

Table 3 shows various cis-regulatory element sequences useful for the invention.

TABLE 3
Endothelial Cell-Specific Cis-regulatory Elements and Promoters

| SEQ ID NOs | Sequences |
|---|---|
| SEQ ID NO: 11 | ctggagggtg actttgcttc tggagccagt acttcatact |
| | tttcatt |
| SEQ ID NO: 12 | aatgaaaagt atgaagtact ggctccagaa gcaaagtcac |
| | cctccag |
| SEQ ID NO: 13 | gtacttcata cttttcattc caatggggtg actttgcttc |
| | tgga |
| SEQ ID NO: 14 | tccagaagca aagtcacccc attggaatga aaagtatgaa |
| | gtac |
| SEQ ID NO: 15 | 3X element |
| | ctccagaagcaaagtcaccccattggaatgaaaagtatgaagtaca |
| | atgaaaagtatgaagt |
| | actggctccagaagcaaagtcaccctccagaagcaaagtcacccca |
| | ttggaatgaaaagtat |
| | gaagtac |
| SEQ ID NO: 16 | 3x element (Complementary Sequence of SEQ ID |
| | NO: 15) |
| | gtacttcatacttttcattccaatggggtgactttgcttctggagg |
| | gtgactttgcttctgg |
| | agccagtacttcatacttttcattgtacttcatacttttcattcca |
| | atggggtgactttgct |
| | tctggag |
| SEQ ID NO: 17 | PPE-1 Promoter |
| | gtacgtgtacttctgatcggcgatactagggagataaggatgtgcc |
| | tgacaaaaccacattg |
| | ttgttgttatcattattatttagttttccttccttgctaactcctg |
| | acggaatctttctcac |
| | ctcaaatgcgaagtactttagtttagaaaagacttggtggaagggg |
| | tggtggtggaaaagta |
| | gggtgatcttccaaactaatctggttccccgcccgccccagtagct |
| | gggattcaagagcgaa |
| | gagtggggatcgtccccttgtttgatcagaaagacataaaaggaaa |
| | atcaagtgaacaatga |
| | tcagccccacctccaccccacccccctgcgcgcgcacaatacaatc |
| | tatttaattgtacttc |
| | atacttttcattccaatggggtgactttgcttctggagaaactctt |
| | gattcttgaactctgg |
| | ggctggcagctagcaaaaggggaagcgggctgctgctctctgcagg |
| | ttctgcagcggtctct |
| | gtctagtgggtgttttctttttcttagccctgcccctggattgtca |
| | gacggcgggcgtctgc |
| | ctctgaagttagccgtgatttcctctagagccgggtcttatctctg |
| | gctgcacgttgcctgt |
| | gggtgactaatcacacaataacattgtttagggctggaatgaagtc |
| | agagctgtttaccccc |
| | actctataggggttcaatataaaaaggcggcggagaactgtccgag |
| | tcagaagcgttcctgc |
| | accggcgctgagagcctgacccggtctgctccgctgtccttgcgcg |
| | ctgcctcccggctgcc |
| | cgcgacgctttcgccccagtggaagggccacttgctgcggccgc |
| SEQ ID NO: 18 | PPE-1-3X promoter |
| | gtacgtgtacttctgatcggcgatactagggagataaggatgtgcc |
| | tgacaaaaccacattg |
| | ttgttgttatcattattatttagttttccttccttgctaactcctg |
| | acggaatctttctcac |
| | ctcaaatgcgaagtactttagtttagaaaagacttggtggaagggg |
| | tggtggtggaaaagta |
| | gggtgatcttccaaactaatctggttccccgcccgccccagtagct |
| | gggattcaagagcgaa |
| | gagtggggatcgtccccttgtttgatcagaaagacataaaaggaaa |
| | atcaagtgaacaatga |
| | tcagccccacctccaccccacccccctgcgcgcgcacaatacaatc |
| | tatttaattgtacttc |
| | atacttttcattccaatggggtgactttgcttctggagaaactctt |
| | gattcttgaactctgg |
| | ggctggcagctagcctccagaagcaaagtcaccccattggaatgaa |
| | aagtatgaagtacaat |
| | gaaaagtatgaagtactggctccagaagcaaagtcaccctccagaa |
| | gcaaagtcaccccatt |
| | ggaatgaaaagtatgaagtacgctagcaaaaggggaagcgggctgc |
| | tgctctctgcaggttc |
| | tgcagcggtctctgtctagtgggtgttttctttttcttagccctgc |
| | ccctggattgtcagac |
| | ggcgggcgtctgcctctgaagttagccgtgatttcctctagagccg |
| | ggtcttatctctggct |
| | gcacgttgcctgtgggtgactaatcacacaataacattgtttaggg |
| | ctggaatgaagtcaga |
| | gctgtttacccccactctataggggttcaatataaaaaggcggcgg |
| | agaactgtccgagtca |
| | gaagcgttcctgcaccggcgctgagagcctgacccggtctgctccg |
| | ctgtccttgcgcgctg |
| | cctcccggctgcccgcgacgctttcgccccagtggaagggccactt |
| | gctgcggccgc |
| SEQ ID NO: 22 | ggtgactttg cttctggag |
| SEQ ID NO: 23 | ctccagaagcaaagtcacc |
| SEQ ID NO: 24 | gtacttcata cttttcatt |
| SEQ ID NO: 25 | aatgaaaagtatgaagtac |
| SEQ ID NO: 26 | Hypoxia Response element |
| | gcacgt |

A cis-regulatory element for the present disclosure can be linked to a promoter upstream or downstream of the promoter or inserted between the two nucleotides in the promoter. The endothelial cell-specific promoter for the present disclosure can utilize any promoters known in the art. For example, suitable promoters which can be utilized for the present disclosure include the endothelial-specific promoters: preproendothelin-1 (PPE-1 promoter), US 2010/0282634, published Nov. 11, 2010; and WO 2011/083464, published Jul. 14, 2011); the PPE-1-3X promoter (U.S. Pat. Nos. 7,579,327, 8,071,740, 8,039,261, US2010/0282634, US 2007/0286845, WO 2011/083464, and WO2011/083466); the TIE-1 (S79347, S79346) and the TIE-2 (U53603) promoters [Sato T N, Proc Natl Acad Sci USA 1993 October 15; 90(20):9355-8], the Endoglin promoter [Y11653; Rius C, Blood 1998 Dec. 15; 92(12):4677-90], the von Willerbrand factor [AF152417; Collins C J Proc Natl Acad Sci USA 1987 July; 84(13):4393-7], the KDR/flk-1 promoter [X89777, X89776; Ronicke V, Circ Res 1996 August; 79(2):277-85], The FLT-1 promoter [D64016 AJ224863; Morishita K, J Biol Chem 1995 Nov. 17; 270(46):27948-53], the Egr-1 promoter [AJ245926; Sukhatme V P, Oncogene Res 1987 Sep.-Oct.; 1(4):343-55], the E-selectin promoter [Y12462; Collins T J Biol Chem 1991 Feb. 5; 266(4):2466-73], The endothelial adhesion molecules promoters: ICAM-1 [X84737; Horley K J EMBO J 1989 Oct.; 8(10):2889-96], VCAM-1 [M92431; Iademarco M F, J Biol Chem 1992 Aug. 15; 267(23): 16323-9], PECAM-1 [AJ313330 X96849; CD31, Newman P J, Science 1990 Mar. 9; 247(4947): 1219-22], the vascular smooth-muscle-specific elements: CArG box X53154 and aortic carboxypeptidase-like protein (ACLP) promoter [AF332596; Layne M D, Circ Res. 2002; 90: 728-736] and Aortic Preferentially Expressed Gene-1 [Yen-Hsu Chen J. Biol. Chem, Vol. 276, Issue 50, 47658-47663, Dec. 14, 2001], all of which are incorporated herein by reference in their entireties.

In one embodiment, a promoter linked to the endothelial cell-specific element comprises a nucleotide sequence at least 60%, 70%, 80%, 90%, 95%, 96%, 97%, 98%, 99%, or 100% of SEQ ID NO: 17, wherein the promoter linked to the element induces endothelial cell-specificity to the gene operably linked to the promoter. In another embodiment, a promoter linked to the endothelial cell-specific element comprises a fragment, a variant, a derivative, or an analog of a wild-type PPE-1 promoter, wherein said fragment, variant, derivative, or analog thereof induces endothelial cell-specificity to the gene operably linked to the promoter. In one example, the endothelial cell-specific element can be inserted between nucleotide residues 442 and 449 corresponding to SEQ ID NO: 17.

In further embodiments, an endothelial cell-specific promoter comprises a hypoxia responsive element. A hypoxia responsive element (EIRE) is located on the antisense strand of the endothelin-1 promoter. This element is a hypoxia-inducible factor-1 binding site that is required for positive regulation of the endothelin-1 promoter (of the human, rat and murine gene) by hypoxia. Hypoxia is a potent signal, inducing the expression of several genes including erythropoietin (Epo), VEGF, and various glycolytic enzymes. The core sequence (8 base pairs) is conserved in all genes that respond to hypoxic conditions and the flanking regions are different from other genes. The ET-I hypoxia responsive element is located between the GAT A-2 and the AP-1 binding sites. In one example, a hypoxia response element comprises SEQ ID NO: 26, a fragment, a variant, a derivative, or an analog thereof.

In other embodiments, an endothelial cell-specific promoter useful for the invention comprises, consists essentially of, or consists of a nucleotide sequence at least 60%, 70%, 80%, 90%, 95%, 96%, 97%, 98%, 99%, or 100% of SEQ ID NO: 18, wherein the promoter linked to the cis-regulatory element induces endothelial cell-specificity to the gene operably linked to the promoter. In another embodiment, an endothelial cell-specific promoter comprises a fragment, a variant, a derivative, or an analog of SEQ ID NO: 18, wherein said fragment, variant, derivative, or analog thereof induces endothelial cell-specificity to the gene operably linked to the promoter.

Additional variations of the endothelial cell-specific promoters can be found at WO2011/083464, WO2011/083466, and WO2012/052423, which are incorporated herein by reference in their entireties.

The present disclosure also provides a novel promoter sequence comprising a nucleotide sequence SEQ ID NO: 17. In one example, the promoter further comprises an endothelial cell-specific cis-regulatory element. In one example, the endothelial cell-specific cis-regulatory element comprises SEQ ID NO: 11, SEQ ID NO: 12, SEQ ID NO: 13, SEQ ID NO: 14, SEQ ID NO: 15, SEQ ID NO: 16, SEQ ID NO: 22, SEQ ID NO: 23, SEQ ID NO: 24, SEQ ID NO: 25, SEQ ID NO: 26 or any fragments, derivatives, variants, or analogs thereof, wherein the fragments, derivatives, variants, or analogs thereof improve endothelial cell-specificity of the promoter compared to a promoter without the cis-regulatory element. In another example, the promoter comprises a nucleotide sequence of SEQ ID NO: 18. The invention includes a nucleic acid construct comprising the novel promoter and a heterologous nucleotide sequence. In one embodiment, the heterologous nucleic acid sequence comprises a nucleotide sequence encoding a Fas-chimera protein described herein. In another embodiment, the heterologous nucleotide sequence comprises an adenovirus sequence.

C. Vector

The present disclosure also provides a vector comprising the nucleic acid construct, which comprises a Fas-chimera gene operably linked to an endothelial cell-specific promoter. For the purposes of this disclosure, numerous vector systems may be employed. For example, various viral gene delivery systems that can be used in the practice of this aspect of the invention include, but are not limited to, an adenoviral vector, an alphavirus vector, an enterovirus vector, a pestivirus vector, a lentiviral vector, a baculoviral vector, a herpesvirus vector, an Epstein Barr viral vector, a papovaviral vector, a poxvirus vector, a vaccinia viral vector, an adeno-associated viral vector and a herpes simplex viral vector.

In another embodiment, a vector comprising a Fas-chimera gene operably linked to an endothelial cell-specific promoter is an adenovirus. For example, the adenovirus can be any one or more of human adenovirus species A (serotypes 12, 18, and 31), B (serotypes 3, 7, 11, 14, 16, 21, 34, 35, 50, and 55), C (serotypes 1, 2, 5, 6, and 57), D (8, 9, 10, 13, 15, 17, 19, 20, 22-30, 32, 33, 36-39, 42-49, 51, 53, 54, and 56), E (serotype 4), F (serotype 40 and 41), or G (serotype 52). In a particular embodiment, the adenovirus for the invention is human adenovirus serotype 5. In some embodiments, the adenovirus useful for gene therapy is a recombinant non-replicating adenovirus, which does not contain an E1 region and an E3 region.

In a particular aspect, the vector is an Ad5-PPE-1-3X-Fas-c vector. In a more particular aspect, the vector is an Ad5-PPE-1-3X-Fas-c vector that comprises, consists essentially of, or consists of SEQ ID NO: 19. In another embodiment, the adenovirus vector is an isolated virus having European Collection of Cell Cultures (ECACC) Accession Number 13021201.

### IV. Treatment Further Comprising One or More Chemotherapeutic Agents

In some aspects of the present disclosure, the post-surgical dose of the vector is administered in combination with one or more chemotherapeutic agents.

A. Chemotherapeutic agents

One or more chemotherapeutic agents that can be administered using the methods of the present disclosure include, but are not limited to, Acivicin; Aclarubicin; Acodazole Hydrochloride; Acronine; Adriamycin; Adozelesin; Aldesleukin; Alimta; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bevacizumab, Bizelesin; Bleomycin Sulfate; Brequinar Sodium; Bropirimine; Busulfan; Cactinomycin; Calusterone; Caracemide; Carbetimer; Carboplatin; Carmustine (BiCNU); Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflornithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Etoposide; Etoposide Phosphate; Etoprine; Fadrozole Hydrochloride; Fazarabine; Fenretinide; Floxuridine; Fludarabine Phosphate; Fluorouracil; Flurocitabine; Fosquidone; Fostriecin Sodium; Gemcitabine; Gemcitabine Hydrochloride; Gliadel® wafer; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-I a; Interferon Gamma-I b; Iproplatin; Irinotecan Hydrochloride; Lanreotide Acetate; Letrozole; Leuprolide Acetate; Liarozole Hydrochloride; Lometrexol Sodium; Lomustine (CCNU); Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; pazotinib; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safingol; Safingol Hydrochloride; Semustine; Simtrazene; Sorafinib; Sparfosate Sodium; Sparsomycin; Spirogermanium Hydrochloride; Spiromustine; Spiroplatin; Streptonigrin; Streptozocin; Sulofenur; Sunitinib; Talisomycin; Taxol; Tecogalan Sodium; Tegafur; Teloxantrone Hydrochloride; Temoporfin; Temozolomide; Teniposide; Teroxirone; Testolactone; Thiamiprine; Thioguanine; Thiotepa; Tiazofuirin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; or Zorubicin Hydrochloride. Additional antineoplastic agents include those disclosed in Chapter 52, Antineoplastic Agents (Paul Calabresi and Bruce A. Chabner), and the introduction thereto, 1202-1263, of Goodman and Gilman's “The Pharmacological Basis of Therapeutics”, Eighth Edition, 1990, McGraw-Hill, Inc.

In some aspects of the disclosure, the one or more chemotherapeutic agents are selected from the group consisting of altretamine, raltritrexed, topotecan, paclitaxel, docetaxel, cisplatin, carboplatin, oxaliplatin, liposomal doxorubicin, gemcitabine, cyclophosphamide, vinorelbine, ifosfamide, etoposide, altretamine, capecitabine, irinotecan, melphalan, pemetrexed, bevacizumab, and albumin bound paclitaxel. In one particular aspect, the chemotherapeutic agent is paclitaxel.

In some aspects, the subject has had up to three, up to two, or up to one previous line of chemotherapy. In other aspects, the subject has not had more than 3 prior lines of chemotherapy for recurrent cancer.

An effective amount of the chemotherapeutic agents is available in the art. In a particular aspect, for example, an effective amount of paclitaxel can be at least about 10 mg/m², at least about 20 mg/m², at least about 30 mg/m², at least about 40 mg/m², at least about 50 mg/m², at least about 60 mg/m², at least about 70 mg/m², at least about 80 mg/m², at least about 90 mg/m², at least about 100 mg/m², or at least about 110 mg/m². In another aspect, an effective amount of paclitaxel is from about 10 mg/m² to about 200 mg/m², from about 20 mg/m² to about 150 mg/m², from about 30 mg/m² to about 100 mg/m², or from 40 mg/m² to about 80 mg/m². In other aspects, an effective amount of paclitaxel is about 10 mg/m², about 20 mg/m², about 30 mg/m², about 40 mg/m², about 50 mg/m², about 60 mg/m², about 70 mg/m², about 80 mg/m², about 90 mg/m², or about 100 mg/m².

In a specific aspect, the post-surgical dose of the vector is administered at an effective amount of 3×10¹² to 3×10¹³ VPs and paclitaxel is administered at an effective amount of 40 mg/m² to 100 mg/m².

In some aspects, the one or more chemotherapeutic agents are repeatedly administered. In particular aspects, the one or more chemotherapeutic agents are repeatedly administered once in about 7 days, once in about 2 weeks, once in about 3 weeks, once in about 4 weeks, once in about 2 months, once in about 3 months, once in about 4 months, once in about 5 months, or once in about 6 months.

B. VEGF Antagonists

In some aspects of the present disclosure, the one or more chemotherapeutic agents are a VEGF antagonist.

VEGF, vascular endothelial growth factor, is an endothelial cell-specific mitogen and an inducer of angiogenesis. The term VEGF encompasses the members of the VEGF gene family: VEGF-A, VEGF-B, VEGF-C, and VEGF-D. VEGF-A is considered the prototype member of the VEGF gene family. Through alternative exon splicing, VEGF-A exists in four different isoforms: VEGF₁₂₁, VEGF₁₆₅, VEGF₁₈₉, and VEGF₂₀₆. The four VEGF-A isoforms are 121, 165, 189, and 206 amino acids in length (respectively) after signal sequence cleavage.

Once expressed, VEGF is secreted extracellularly where it binds to the extracellular region of a VEGF receptor (VEGFR). There are two primary VEGFRs, VEGFR-1 or VEGFR-2, both of which are receptor tyrosine kinases. A third VEGFR, VEGFR-3, is a related receptor tyrosine kinase that only binds VEGF-C and VEGF-D. Upon binding to VEGF, the VEGFRs signal downstream events that lead to endothelial cell proliferation and angiogenesis. VEGF-C and VEGF-D are known to regulate lymphatic angiogenesis.

The VEGF gene contains nucleotide sequences that are highly homologous to those of hypoxia-inducible factor-1 (HIF-1). These HIF-1 like sequences enable induction of VEGF gene expression under hypoxic conditions. Thus, under low oxygen conditions, such as within a tumor microenvironment, VEGF gene expression is induced. The production of high levels of VEGF within a tumor bed results in increased VEGFR signaling and thus endothelial cell growth and angiogenesis. The formation of new blood vessels within the tumor provides blood and oxygen to the growing tumor.

Due to the prominent role of VEGF in angiogenesis and tumor growth and development, VEGF antagonists are studied as potential cancer therapeutic agents. VEGF antagonists can prevent VEGF activity by binding directly to VEGF and blocking its interaction with a VEGFR. This reduces signaling from the VEGFR and downstream events, thereby causing a reduction in angiogenesis. In some aspects of the present disclosure, a VEGF antagonist useful in the methods is an anti-VEGF antibody or a VEGF binding molecule. In some aspects of the disclosure, an anti-VEGF antibody or VEGF-binding molecule is a monoclonal antibody, a humanized antibody, a human antibody, a single chain antibody, or a chimeric antibody. In some aspects, an anti-VEGF antibody or VEGF-binding molecule for the therapy comprises Fab, F(ab)₂, Fv, or scFv.

Another type of VEGF antagonist that can reduce or inhibit VEGF activity is a molecule binding to a VEGFR and thus blocking VEGFR interaction with VEGF. This interference of receptor/ligand binding prevents VEGFR signaling and reduces angiogenesis and endothelial cell proliferation. In some aspects of the present disclosure, the VEGF antagonist is an anti-VEGFR antibody or VEGFR-binding molecule. In some aspects, the anti-VEGFR antibody or VEGFR-binding molecule is a monoclonal antibody, a humanized antibody, a human antibody, a single chain antibody, or a chimeric antibody. In some aspects, the anti-VEGFR antibody or VEGFR-binding molecule comprises Fab, F(ab)₂, Fv, or scFv.

VEGF antagonists that bind to VEGF or VEGFR can inhibit VEGF activity by similar mechanisms of action in that they prevent receptor/ligand interaction, VEGFR signaling, and downstream signaling events such as endothelial cell proliferation and angiogenesis. Thus, in some aspects of the disclosure, the VEGF antagonist is selected from the group consisting of bevacizumab (U.S. Pat. No. 7,169,901, incorporated herein by reference in its entirety), ranibizumab (U.S. Pat. No. 7,297,334, incorporated herein by reference in its entirety), VGX-100 (U.S. Pat. No. 7,423,125, incorporated herein by reference in its entirety), r84 (U.S. Pat. No. 8,034,905, incorporated herein by reference in its entirety), aflibercept (U.S. Pat. No. 5,952,199, incorporated herein by reference in its entirety), IMC-18F1 (U.S. Pat. No. 7,972,596, incorporated herein by reference in its entirety), IMC-1C11 (PCT/US2000/02180, incorporated herein by reference in its entirety), and ramucirumab (U.S. Pat. No. 7,498,414, incorporated herein by reference in its entirety). A VEGF binding molecule includes other forms of antibody derived molecules, e.g., a monobody, diabody, minibody, or any chimeric proteins comprising at least one CDR of a VEGF binding antibody, e.g., bevacizumab.

In particular aspects, the anti-VEGF antibody or VEGF binding molecule comprises at least one CDR selected from the group consisting of V_{H} CDR1 (SEQ ID NO: 28), V_{H} CDR2 (SEQ ID NO: 29), V_{H} CDR3 (SEQ ID NO: 30), V_{L} CDR1 (SEQ ID NO: 31), V_{L} CDR2 (SEQ ID NO: 32), V_{L} CDR3 (SEQ ID NO: 33), and any combination thereof. See Table 4.

TABLE 4
Amino Acid Sequences of Complementarity
Determining Regions

| CDR | Sequence |
|---|---|
| V_{H} CDR1 (SEQ ID NO: 28) | GYTFTNYGMN |
| V_{H} CDR2 (SEQ ID NO: 29) | WINTYTGEPTYAADFKR |
| V_{H} CDR3 (SEQ ID NO: 30) | YPHYYGSSHWYFDV |
| V_{L} CDR1 (SEQ ID NO: 31) | SASQDISNYLN |
| V_{L} CDR2 (SEQ ID NO: 32) | FTSSLHS |
| V_{L} CDR3 (SEQ ID NO: 33) | QQYSTVPWT |

In particular aspects, the anti-VEGF antibody or the VEGF binding molecule comprises CDR1 (SEQ ID NO: 28), CDR2 (SEQ ID NO: 29), or CDR3 (SEQ ID NO: 30) of the heavy chain variable region (V_{H}) of bevacizumab. For example, an anti-VEGF antibody or VEGF binding molecule comprises CDR1 and CRD2 of V_{H}, CDR 1 and CDR3 of V_{H}, CDR2 and CDR3 of V_{H}, or CDR1, CDR2, or CDR3 of V_{H}. In particular aspects, the anti-VEGF antibody or the VEGF binding molecule comprises CDR1 (SEQ ID NO: 31), CDR2 (SEQ ID NO: 32), or CDR3 (SEQ ID NO: 33) of the light chain variable region (V_{L}) of bevacizumab. For example, an anti-VEGF antibody or VEGF-binding molecule comprises CDR1 and CDR2 of V_{L}, CDR1 and CDR3 of V_{L}, CDR2 and CDR3 of V_{L}, or CDR1, CDR2, and CDR3 of V_{L}. In some embodiments, an anti-VEGF antibody or VEGF binding molecule comprises V_{H} of bevacizumab. In certain embodiments, an anti-VEGF antibody or VEGF binding molecule comprises V_{L} of bevacizumab.

In another aspect of the present disclosure, the anti-VEGF antibody or VEGF binding molecule comprises V_{H} CDR1 (SEQ ID NO: 28), V_{H} CDR2 (SEQ ID NO: 29), V_{H} CDR3 (SEQ ID NO: 30), V_{L} CDR1 (SEQ ID NO: 31), V_{L} CDR2 (SEQ ID NO: 32), and V_{L} CDR3 (SEQ ID NO: 33).

An effective amount of the VEGF antagonist is available in the art. For example, the dose of the VEGF antagonist (e.g., bevacizumab) can be measured in mg/kg body weight. In one aspect, for example, an effective amount of bevacizumab can be at least about 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, or 15 mg/kg. In some aspects, an effective amount of bevacizumab include equal to or less than about 15 mg/kg, 14 mg/kg, 13 mg/kg, 12 mg/kg, 11 mg/kg, 10 mg/kg, 9 mg/kg, 8 mg/kg, 7 mg/kg, 6 mg/kg, 5 mg/kg, 4 mg/kg, 3 mg/kg, 2 mg/kg, or 1 mg/kg. In one aspect, the dose of bevacizumab administered in the combination with the post-surgical dose of the vector is lower than the dose of bevacizumab without the vector (e.g., a therapy using bevacizumab alone).

In a specific aspect, the post-surgical dose of the vector is administered at an effective amount of 3×10¹² to 3×10¹³ VPs and bevacizumab is administered at an effective amount of 5 mg/kg to 15 mg/kg.

In some aspects, the VEGF antagonist (e.g., bevacizumab) is repeatedly administered. In particular aspects, the bevacizumab is repeatedly administered once in about 7 days, once in about 2 weeks, once in about 3 weeks, once in about 4 weeks, once in about 2 months, once in about 3 months, once in about 4 months, once in about 5 months, or once in about 6 months. In a more particular aspect, the bevacizumab is repeatedly administered once in about 2 weeks.

The present disclosure provides methods of inducing or improving anti-tumor response in a subject having a tumor comprising administering to the subject a priming dose of a vector, wherein the priming dose of the vector is administered prior to surgical removal of the tumor or a portion thereof; surgically removing the tumor or a portion thereof; and administering to the subject a post-surgical dose of the vector in combination with bevacizumab.

The present disclosure also provides methods for reducing or inhibiting the size of a tumor in a subject comprising administering to the subject a priming dose of a vector, wherein the priming dose of the vector is administered prior to surgical removal of the tumor or a portion thereof; surgically removing the tumor or a portion thereof; and administering to the subject a post-surgical dose of the vector in combination with bevacizumab.

The present disclosure also provides methods for inhibiting or reducing angiogenesis in a subject having a tumor comprising administering to the subject a priming dose of a vector, wherein the priming dose of the vector is administered prior to surgical removal of the tumor or a portion thereof; surgically removing the tumor or a portion thereof; and administering to the subject a post-surgical dose of the vector in combination with bevacizumab.

The present disclosure also provides methods for inducing apoptosis of an endothelial cell in a tumor of a subject comprising administering to the subject a priming dose of a vector, wherein the priming dose of the vector is administered prior to surgical removal of the tumor or a portion thereof; surgically removing the tumor or a portion thereof; and administering to the subject a post-surgical dose of the vector in combination with bevacizumab.

The present disclosure also provides methods for treating a disease or condition associated with a tumor in a subject comprising administering to the subject a priming dose of a vector, wherein the priming dose of the vector is administered prior to surgical removal of the tumor or a portion thereof; surgically removing the tumor or a portion thereof; and administering to the subject a post-surgical dose of the vector in combination with bevacizumab.

In some aspects of the present disclosure, the regimen used for administering the post-surgical dose of the vector and the bevacizumab comprises repeated administration of the vector and the bevacizumab. In one aspect, the vector is repeatedly administered every day, once in about 2 days, once in about 3 days, once in about 4 days, once in about 5 days, once in about 6 days, once in about 7 days, once in about 2 weeks, once in about 3 weeks, once in about 4 weeks, once in about 5 weeks, once in about 6 weeks, once in about 7 weeks, once in about 2 months, or once in about 6 months. In another aspect, the bevacizumab is repeatedly administered once in about 7 days, once in about 2 weeks, once in about 3 weeks, once in about 4 weeks, once in about 2 months, once in about 3 months, once in about 4 months, once in about 5 months, or once in about 6 months. In a particular aspect, the vector is administered every 2 months and bevacizumab is administered every 2 weeks.

### V. Pharmaceutical Compositions

Also provided in the invention is a pharmaceutical composition comprising a vector expressing a Fas-chimera protein used in the methods of the invention. The pharmaceutical composition can be formulated for administration to mammals, including humans. The pharmaceutical compositions used in the methods of this invention comprise pharmaceutically acceptable carriers, including, e.g., ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium tri silicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. In one embodiment, the composition is formulated by adding saline.

The compositions of the present disclosure may be administered by any suitable method, e.g., parenterally (e.g., includes subcutaneous, intravenous, intramuscular, intra-articular, intra-synovial, intrasternal, intrathecal, intrahepatic, intralesional and intracranial injection or infusion techniques), intraventricularly, orally, by inhalation spray, topically, rectally, nasally, buccally, vaginally or via an implanted reservoir. In one embodiment, the combination therapy is delivered systemically or locally. For systemic or local delivery, the pharmaceutical formulation can be administered using a mechanical device such as a needle, cannula or surgical instruments.

Sterile injectable forms of the compositions used in the methods of this invention may be aqueous or oleaginous suspension. These suspensions may be formulated according to techniques known in the art using suitable dispersing or wetting agents and suspending agents. The sterile, injectable preparation may also be a sterile, injectable solution or suspension in a non-toxic parenterally acceptable diluent or solvent, for example as a suspension in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Parenteral formulations may be a single bolus dose, an infusion or a loading bolus dose followed with a maintenance dose. These compositions may be administered at specific fixed or variable intervals, e.g., once a day, or on an “as needed” basis.

Certain pharmaceutical compositions used in the methods of this invention may be orally administered in an acceptable dosage form including, e.g., capsules, tablets, aqueous suspensions or solutions. Certain pharmaceutical compositions also may be administered by nasal aerosol or inhalation. Such compositions may be prepared as solutions in saline, employing benzyl alcohol or other suitable preservatives, absorption promoters to enhance bioavailability, and/or other conventional solubilizing or dispersing agents.

### EXAMPLES

### Example 1

This example provides a randomized, controlled phase II surgical trial to evaluate early immunologic pharmacodynamic parameters for the viral cancer therapy Ad5-PPE-1-3X-Fas-c in patients with surgically accessible recurrent/progressive glioblastoma (GBM).

### Objectives:

### Study Design

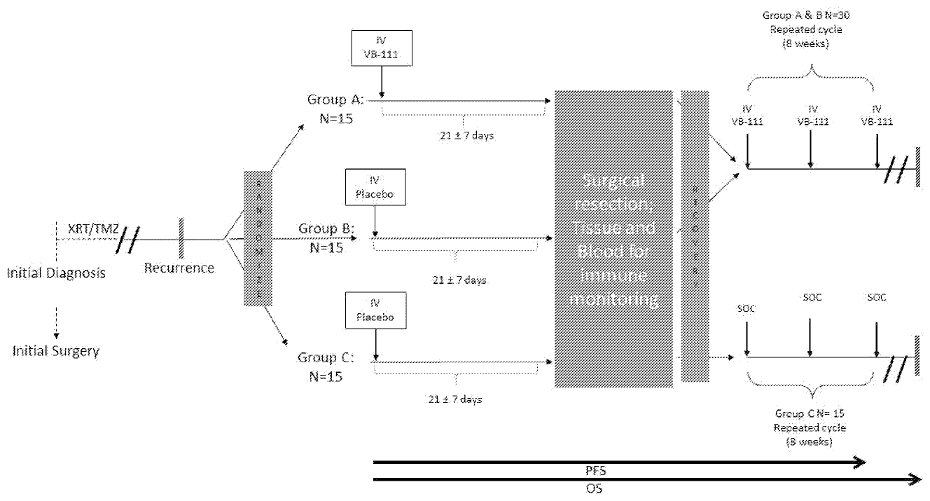

This is a randomized, controlled, blinded, phase II, surgical trial to evaluate early immunologic pharmacodynamic parameters for an Ad5-PPE-1-3X-Fas-c viral cancer therapy, ofranergene obadenovec (VB-111), in participants with surgically accessible recurrent/progressive glioblastoma (rGBM). The principal goal of this study is to investigate if neoadjuvant VB-111 elicits a tumoral immunologic response, if the immune response can be sensitively monitored using tumor and peripheral blood immune cells, and if neoadjuvant VB-111 increases tumor-specific T cells. See FIG. 1.

Group A: VB-111 at 1×10¹³ VPs will be administered intravenously 21±7 days prior to surgery. Upon recovering from surgery (within 28-35 days after surgery), participants will receive intravenous VB-111 every 6 weeks. Upon evidence of contrast-enhancing progression, participants may initiate bevacizumab as needed for supportive care and will continue with VB-111 infusions until progression is supported by two consecutive time points of tumor growth.

Group B: Placebo will be administered intravenously 21±7 days prior to surgery. Upon recovering from surgery (within 28-35 days after surgery), participants will receive intravenous VB-111 every 6 weeks. Upon evidence of contrast-enhancing progression, participants may initiate bevacizumab as needed for supportive care and will continue with VB-111 infusions until progression is supported by two consecutive time points of tumor growth.

Group C: Placebo will be administered intravenously 21±7 days prior to surgery. Upon recovery from surgery, participants will receive standard of care treatment and every 6 weeks until evidence of progression is supported by two consecutive time points of tumor growth.

After recovering from surgery, participants will be evaluated every 6 weeks with radiographic imaging to assess response to treatment. The Response Assessment in Neuro-Oncology (RANO) criteria will be used as the efficacy endpoint of response rate. A modified RANO (iRANO) (as described in Section 11.4) will be used to evaluate response and progression in an exploratory fashion due to the tumor response patterns seen with immunotherapy treatment. Adverse events will be monitored throughout the trial and graded in severity according to the guidelines outlined in the NCI Common Terminology Criteria for Adverse Events (CTCAE) version 5.0. Treatment with study therapy will continue until progression is supported by two consecutive time points of tumor growth, unacceptable adverse event(s), intercurrent illness that prevents further administration of treatment, investigator's decision to withdraw the participant, participant withdraws consent, pregnancy of the participant, noncompliance with trial treatment or procedure requirements, completion of 24 months of study therapy, or administrative reasons.

This study will define the neoadjuvant use of VB-111, leading to: 1) increased tumor infiltrating T lymphocytes within the tumor; and 2) enhanced systemic specific T cell responses. The combination of tumor tissue, peripheral blood, and imaging evaluations will be assessed.

### Primary Objectives

Primary Objective 1: To evaluate the influence of VB-111 on tumor infiltrating T lymphocyte (TIL) density in recurrent/progressive GBM participants.

The neoadjuvant VB-111 will increase tumor infiltrating T lymphocytes within the tumor and enhance systemic tumor-specific T cell responses compared to adjuvant and control tumors.

Primary Objective 2: To evaluate the safety and tolerability of intravenous VB-111 in progressive/recurrent GBM participants undergoing surgery.

Safety analysis will be based on toxicities and grades as defined by CTCAE version 5.0 criteria, including serious adverse events (SAEs) and events of clinical interest (ECIs). The attribution to drug, time-of-onset, duration of the event, resolution, and any concomitant medications administered will be recorded. AEs analyzed include but are not limited to all AEs, SAEs, and fatal AEs.

### Secondary Objectives

Secondary Objective 1: To estimate the 6-month progression-free survival (PFS6) in recurrent/progressive GBM participants treated with VB-111 (Group A and Group B) compared to control (Group C), using RANO criteria. It is expected that a greater percentage of participants who are treated with VB-111 will not have experienced tumor progression at 6 months after treatment

Secondary Objective 2: To calculate the overall survival of recurrent/progressive GBM participants in each arm. It is expected that progressive/recurrent GBM participants treated with VB-111 will live significantly longer than Group C and/or historical controls of this participant population.

Secondary Objective 3: To evaluate the influence of VB-111 on peripheral T cell responses, specifically on expanded TCR clones. It is expected that specific subsets of TCR clones will expand in response to VB-111.

### Exploratory Objectives

To evaluate the associations between exploratory biomarkers, clinical outcomes and adverse events which include:
Exploring the influence of VB-111 on cell cycle-related genetic signatures or IFN-γ associated signatures within the tumor microenvironment, and correlating with clinical responses.
Exploring the influence of VB-111 on oligoclonal T-cell populations within tumor tissue and peripheral blood, and correlating with clinical responses.
Exploring the influence of VB-111 on specific MRI parameters, and correlating with tumor and peripheral blood immune responses.
Estimating the efficacy of VB-111 by PFS and OS as defined by RANO.
Estimating the efficacy of VB-111 by PFS6, PFS and OS as defined by iRANO.

Participant Selection

Inclusion Criteria

The following will be inclusion criteria:

Histologically confirmed diagnosis of glioblastoma;

First or second progression of glioblastoma/gliosarcoma (according to RANO criteria) following standard of care treatment upon initial diagnosis with radiation;

Measurable disease by RANO criteria at progression;

The maximal tumor volume at baseline meets the following criteria: Longest diameter≤4 cm;

Surgically resectable disease at progression;

An interval of the following durations prior to randomization:
At least 28 days from prior surgical resection, or 7 days from stereotactic biopsy;
At least 12 weeks from prior radiotherapy, unless there is unequivocal histologic confirmation of tumor progression;
At least 23 days from prior chemotherapy;
At least 42 days from nitrosureas;
At least 42 days from other anti-tumor therapies (including vaccines);
At least 28 days from any investigational agent.

Corticosteroid use at or less than dexamethasone 2 mg daily. Participants should be on a stable or decreasing dose for at least 1 week prior to randomization, with no anticipation of a need to increase the steroid dose throughout the study;

Age≥18;

KPS≥70%;

Life expectancy of at least 3 months;

Adequate bone marrow, liver, and renal function according to the following criteria:
Absolute neutrophil count 1,500 cells/mL
Platelets≥100,000 cells/mL.
Total bilirubin within upper limit of normal (ULN) OR Direct bilirubin≤institutional

ULN for subjects with total bilirubin levels>1.5 institutional ULN.
Aspartate aminotransferase (AST)≤2.0×ULN.
Serum creatinine level≤ULN or creatinine clearance≥50 mL/min for participants with creatinine levels above normal limits (calculated by the Cockcroft-Gault formula);
Ability to understand and willingness to sign a written informed consent document;
Males and females of childbearing potential must utilize a standard contraception method throughout the course of the trial;

Women of childbearing potential must have a negative serum beta-human chorionic gonadotropin pregnancy test at initial screening and/or within 7 days prior to Day 1;

Women of childbearing potential and men with female spouses of childbearing potential must agree to use two methods of reliable contraception simultaneously or to practice complete abstinence from heterosexual contact prior to study entry, while receiving treatment, and for 4 months after undergoing treatment. One method must include a highly effective method such as an intrauterine device, hormonal (birth control pills, injections or implants), tubal ligation or partner's vasectomy. The other method can be an additional hormonal therapy or barrier method such as a male condom, diaphragm or cervical cap. Should a woman become pregnant or suspect she is pregnant while she or her partner is participating in the study, she should inform her treating physician immediately.

A woman is considered not to be of childbearing potential if she is postmenopausal, defined by amenorrhea of 12 months duration and age 45, or has undergone hysterectomy and/or bilateral oophorectomy.

### Exclusion Criteria

The following are exclusion criteria:

Current or planned participation in a study of an investigational agent or using an investigational device. Has tumor primarily localized to the brainstem or spinal cord.

Has presence of diffuse leptomeningeal disease or extracranial disease.

Surgical procedure (including open biopsy, surgical resection, wound revision, or any other major surgery involving entry into a body cavity) or significant traumatic injury within 28 days prior to first study treatment.

Minor surgical procedure (e.g. stereotactic biopsy or shunt placement) within 7 days of first study treatment, placement of vascular access within 2 days of first study treatment.

Expected to have surgery other than the neurosurgical procedure intended for the GBM lesion during study treatment period.

Prior stereotactic radiotherapy (Note: those who have had biopsy proven tumor recurrence at a site of SRS treatment should be considered eligible if approved by the study central Investigator).

Prior anti-angiogenic therapy including VEGF sequestering agents (i.e. bevacizumab, aflibercept, etc.) or VEGFR inhibitors (cedirinib, pazopanib, sunitinib, sorafenib, etc.).

Prior administration of the study drug VB-111.

Concomitant medication that may interfere with study results (e.g. immunosuppressive agents other than inhaled, topical or intra-articular steroids or a stable or decreasing dose of oral corticosteroids of up to <2 mg/day dexamethasone equivalent).

Known active second malignancy. Exceptions include non-melanoma skin cancers, non-metastatic prostate cancer, in situ cervical cancer, and ductal or lobular carcinoma in situ of the breast. Participants are not considered to have currently active malignancy if they have completed anticancer therapy and have been disease free for greater than 2 years prior to screening.

Uncontrolled intercurrent illness including but not limited to ongoing or active infection, symptomatic congestive heart failure, unstable angina pectoris, cardiac arrhythmia, or psychiatric illness/social situations that would limit compliance with study requirements.

History of stroke or transient ischemic attack within 6 months prior to randomization.

Evidence of CNS hemorrhage CTCAE grade 2 or above on screening MRI.

Active cardiac disease within 6 months prior to randomization (i.e. acute coronary syndrome, unstable angina, New York Heart Association grade II or greater congestive heart failure, or serious cardiac arrhythmia uncontrolled by medication or potentially interfering with protocol treatment).

Significant vascular disease within 6 months prior to randomization (e.g. aortic aneurysm requiring surgical repair, peripheral arterial thrombosis, symptomatic peripheral vascular disease).

History of venous thromboembolism CTCAE version 5.0 grade 3 or greater.

Known proliferative and/or vascular retinopathy.

Inadequately controlled hypertension (defined as systolic blood pressure>150 mmHg and/or diastolic blood pressure>100 mmHg) within 1 week of randomization.

History of pulmonary hemorrhage/hemoptysis grade≥2 (defined as ≥2.5 mL bright red blood per episode) within 6 months of randomization.

History of active gastrointestinal bleeding within 6 months prior to randomization.

History or evidence of inherited bleeding diathesis or significant coagulopathy at risk of bleeding (i.e. in the absence of therapeutic anticoagulation).

Current or recent (within 10 days of study randomization) use of aspirin>325 mg/day, clopidogrel>75 mg/day or equivalent. Therapeutic or prophylactic use of anticoagulants is allowed.

Known liver disease (alcoholic, drug/toxin induced, genetic or autoimmune).

History of gastrointestinal perforation or abscess.

Positive testing to any of the following viruses: HIV, HBV, HCV within the last 6 months. Exceptions include participants with serology positive for HBV indicating past exposure but without evidence of active infection (e.g. negative PCR).

History of intracranial abscess within 6 months prior to randomization.

Serious non-healing wound, active ulcer, or untreated bone fracture.

Pregnant or breastfeeding participants.

Study Plan

This is a randomized, controlled, blinded, phase II, surgical trial to define early immunologic pharmacodynamic parameters for the viral cancer therapy VB-111 in participants with surgically accessible recurrent/progressive glioblastoma (rGBM).

Participants will be screened for eligibility up to 14 days prior to randomization at the baseline visit. They will be sequentially randomized to one of three groups in a 1:1:1 ratio of the Neoadjuvant/Adjuvant arm (Group A), Adjuvant arm (Group B), and Control arm (Group C).

Group A (Neoadjuvant/adjuvant): Fifteen participants will receive VB-111 at 1×10¹³ VPs intravenously 21±7 days prior to surgery. Tumor samples will be obtained at time of surgery, and tissue (fresh, frozen and FFPE) will be processed to achieve primary, secondary and exploratory objectives. Upon recovering from surgery (within 28-35 days after surgery), participants will resume single agent intravenous VB-111 every 6 weeks until tumor progression is supported by two consecutive time points of tumor growth (based on RANO criteria) or adverse event requiring discontinuation of study drug. Upon initial evidence of contrast-enhancing progression, and if clinically stable, participants may continue with VB-111 infusions. Bevacizumab may be used as supportive care if clinically indicated. Blood samples will be obtained as pharmacodynamics markers throughout the study. Dose holds and symptomatic management will occur based on preset adverse event determination. DLTs will not be determined. The toxicity evaluation period will begin with registration and extend to 30 days after the last treatment day. Participants will be followed for MM changes, clinical exam and steroid doses from the registration period until the second progression. After tumor progression is confirmed on the second consecutive MRI, VB-111 will stop and participants will be followed every 3 months for vital status until death.

Group B (Adjuvant): Fifteen participants will receive Placebo intravenously 21±7 days prior to surgery. Tumor samples will be obtained at time of surgery, and tissue (fresh, frozen and FFPE) will be processed to achieve primary, secondary and exploratory objectives. Upon recovering from surgery (within 28-35 days after surgery), participants will receive single agent intravenous VB-111 every 6 weeks until tumor progression is supported by two consecutive time points of tumor growth (based on RANO criteria) or adverse event requiring discontinuation of study drug. Upon evidence of contrast-enhancing progression, and if clinically stable, participants may continue with VB-111 infusions. Bevacizumab may be used as supportive care if clinically indicated. Blood samples will be obtained as pharmacodynamics markers throughout the study. Dose holds and symptomatic management will occur based on preset adverse event determination. DLTs will not be determined. The toxicity evaluation period will begin with registration and extend to 30 days after the last treatment day. Participants will be followed for MM changes, clinical exam and steroid doses from the registration period until the second progression. After tumor progression is confirmed on the second consecutive MM, VB-111 will stop and participants will be followed every 3 months for vital status until death.

Group C (Control): Fifteen participants will receive Placebo intravenously 21±7 days prior to surgery. Tumor samples will be obtained at time of surgery, and tissue (fresh, frozen and FFPE) will be processed to achieve primary, secondary and exploratory objectives. Upon recovering from surgery (within 28-35 days after surgery), participants will receive standard of care treatment until evidence of progression as determined in Group A and Group B.

In some aspects of the disclosure, any of the single agent treatments for participants randomized to the standard of care arm listed in Table 5 can be used for the control treatment arm.

TABLE 5
Single Agent Standard of Care Treatments.

| Single Agent | |
|---|---|
| Treatment | Dose |
| Lomustine | (Oral [PO]) 110 mg/m2 every 6 weeks is suggested |
| Temozolomide | (PO or IV) Initial dose of 150 mg/m2 once daily for |
| | 5 consecutive days per 28-day treatment cycles, may be |
| | increased to 200 mg/m2 once daily for 5 consecutive |
| | days in the following 28-day treatment cycles |
| Temozolomide | (PO) 50 mg/m2 once daily continuously |
| metronomic | |
| Bevacizumab | (IV) 10 mg/kg every 2 weeks |

After recovering from surgery, participants will be evaluated every 6 weeks with radiographic imaging to assess response to treatment. The Response Assessment in Neuro-Oncology (RANO) criteria will be used as the efficacy endpoint of response rate. A modified RANO (iRANO) will be used to evaluate response and progression in an exploratory fashion due to the tumor response patterns seen with immunotherapy treatment (e.g., tumor flare). Adverse events will be monitored throughout the trial and graded in severity according to the guidelines outlined in the NCI CTCAE version 5.0. Treatment with study therapy will continue until documented disease progression supported by two consecutive time points of tumor growth, unacceptable adverse event(s), intercurrent illness that prevents further administration of treatment, investigator's decision to withdraw the participant, participant withdraws consent, pregnancy of the participant, noncompliance with trial treatment or procedure requirements, completion of 24 months of study therapy, or administrative reasons.

### Dose Modification

For patients who experience an adverse event related to VB-111 and are scheduled for a repeat dose, the repeat dose can be delayed until the severity of the event has improved to CTCAE Grade 1 or better. No dose modifications are allowed.

For patients who experience an adverse event related to standard of care and are scheduled for a repeat dose, the repeat dose can be delayed until the severity of the event has improved to CTCAE Grade 1 or better. Dose reductions are permitted for standard of care therapies.

Dosing interruptions are permitted in the case of medical/surgical events or logistical reasons not related to study therapy (e.g., elective surgery, unrelated medical events, participant vacation, and/or holidays). Participants should be placed back on study therapy within 3 weeks of the scheduled interruption, unless otherwise discussed with the Sponsor. The reason for interruption should be documented in the participant's study record.

Criteria for Further Dosing with Study Drug

Recovered to Grade 1 or less from the toxic effects of earlier study-related intervention.

Adequate bone marrow, liver, and renal function according to the following criteria:
Absolute neutrophil count≥1,500 cells/mL.
Platelets≥100,000 cells/mL.
Total bilirubin within upper limit of normal (ULN).
Aspartate aminotransferase (AST)≤2.0×ULN.
Serum creatinine level≤ULN or creatinine clearance≥50 mL/min for participants with creatinine levels above normal limits (calculated by the Cockcroft-Gault formula).

For women, a negative pregnancy test.

No intercurrent illness or medical condition that, in the opinion of the Investigator, may cause excess risk for the participant's continued treatment with the study drug.

In any case of doubt or failure to meet the criteria listed above, the investigator should contact the Medical Monitor for instructions on how to proceed with the treatment.

### Timing of Dose Administration

Priming Dose Administration

VB-111/placebo must be administrated within 5 days from randomization.

Group A: Participants will receive VB-111 1×10¹³ VPs by intravenous infusion 21±7 days prior to scheduled surgical resection.

Group B: Participants will receive placebo by intravenous infusion 21±7 days prior to scheduled surgical resection.

Group C: Participants will receive placebo by intravenous infusion 21±7 days prior to scheduled surgical resection.

### Post-Surgery Dose Administration

For Group A and Group B, upon recovery from surgery (within 28-35 days after surgery), trial treatment should be administered after all procedures/assessments have been completed and reviewed. Trial treatment may be administered up to 3 days before or after the scheduled infusion/injection day of each cycle due to administrative reasons.

All trial treatments will be administered on an outpatient basis.

Group A: Participants will resume VB-111 every 6 weeks

Group B: Participants will receive VB-111 every 6 weeks

Group C: Participants will receive standard of care treatments

Duration of Therapy and Criteria for Removal from Study Treatment

Duration of therapy will depend on individual response, evidence of disease progression and tolerance. Treatment may continue for up to 24 months or until one of the following criteria applies:

The participant or legal representative (such as a parent or legal guardian) withdraws consent;

Radiographic tumor progression is supported by two consecutive time points of tumor growth;

Unacceptable adverse experiences;

Intercurrent illness that prevents further administration of treatment;

Investigator's decision to withdraw the participant;

The participant has a confirmed positive serum pregnancy test;

Noncompliance with trial treatment or procedure requirements;

General or specific changes in the participant's condition render the participant unacceptable for further treatment in the judgment of the treating Investigator; or Administrative reasons.

Participants will be removed from the protocol therapy when any of these criteria apply. The reason for removal from protocol therapy and the date the participant was removed must be documented in the case report form (CRF). Alternative care options will be discussed with the participant.

In the event of unusual or life-threatening complications, treating Investigators must immediately notify the Overall Clinical Principle Investigator.

### End of Treatment Evaluation and Follow-Up

After the end of treatment, each participant will be followed for 30 days for adverse event monitoring and 90 days for serious adverse event reporting.

Participants removed from protocol therapy for unacceptable adverse events will be followed until resolution or stabilization of the adverse event.

Participants who discontinue for reasons other than progressive disease will have post-treatment follow-up for disease status until disease progression, initiating a non-study cancer treatment, withdrawing consent or becoming lost to follow-up.

### VB-111 Preparation

TABLE 6
VB-111 Preparation

| | | | | Take this | | | Syringe | | Vol. to inject | Vol. to inject |
|---|---|---|---|---|---|---|---|---|---|---|
| | Conc. | | # Vials | vol. of | Syringe | | type | | participant | participant |
| Dose | in vial | Vol. of | of | VB-111 | type for | Vol. of | for | Total | weight | weight |
| (VPs) | (VP/ml) | VB-111 | VB-111 | (ml) | VB-111 | saline | saline | vol. | ≥ 50 Kg | < 50 Kg |
| 1 × 10¹³ | 10¹² | 5 mL | 2 | 2 × 5 mL | 10 ml | 40 mL | 50* | 50 ml | (50 mL) | 35 mL** |

*The pharmacist can either use a sterile empty bag and individually add 40 ml NS + 10 ml VB-111 to the bag; or use a 50 ml bag of NS and remove the excess volume then add the VB-111.
**35 ml for participants < 50 kg represents a 30% reduction of VB-111.

As VB-111 is included in risk group 2, all work should be done in BSL II conditions. The entire process of drug preparation shall be carried out at room temperature in a biosafety cabinet (BSC). After thawing, the drug should be diluted in room temperature saline, as soon as possible. Note that if needed, the drug may be maintained on ice for up to 3 hours before the dilution. Once the drug is in its final formulation in saline, keep at room temperature until administration. The maximum time for VB-111/placebo in saline (until completion of administration) is 60 minutes (plus a 30-minute window) at room temperature

The site member preparing the drug shall verify that the information on the container is appropriate for the study and for the participant: product name, concentration, batch number.

Determine the volume to be applied according to the participant's weight (see Table 3).

For patients who weigh>50 kg: Place 40 ml saline (brought to room temperature) in a 50 ml sterile syringe. Alternatively, select a 50 ml saline bag and remove the excess volume (10 ml).

For patients who weigh<50 kg: Place 28 ml saline (brought to room temperature) in a 50 ml sterile syringe.

Thaw two vials of VB-111 solution at room temperature. Rubbing between gloved hands may be used to shorten the process. Be sure to mark the time of thaw.

For patients who weigh>50 kg: Using a 10 ml syringe, pull 5 ml of VB-111 from each of the vials intended for the specific participant. For patients who weigh<50 kg pull 5 ml from the first vial and only 2 ml from the second vial.

Add VB-111 to the syringe/saline bag containing the saline solution prepared in advance. Draw the piston to mix the remaining VB-111 in the syringe with saline and push it back into the syringe/saline bag.

Mix the diluted drug by swirling the contents by hand.

Any Standard Closed system drug-transfer device (CSTD) may be used for drug preparation and administration.

After completing the preparation, perform a reconciliation process:

Check that the correct number of source vials was used.

Record vials assigned to the participant in the drug accountability log.

After preparation of the drug solution, clean the drug formulation area in the pharmacy according to the pharmacy procedures and MSDS. Remaining material used for clinical purposes is collected in their original container and disposed as detailed under the MSDS.

The preparation of the Neoadjuvant dose of VB-111/placebo will be performed by an unblinded site pharmacist or designee who is not involved in other study activities.

Participants who weigh less than 50 kg will receive VB-111/placebo at a reduced dose of 0.7×10¹³ VPs in 35 mL instead of 1×10¹³ VPs in 50 mL. VB-111 should be prepared and administered as follows: 2 vials of VB-111 should be thawed. 7 mL of VB-111 should be taken from the vials (5 ml from the first vial and 2 ml from the second vial) and combined with 28 mL of saline to make a total volume of 35 mL. Total dose of 0.7×10¹³ represents a 30% reduction of VB-111 dose.

### Placebo

Normal Saline (NS) will be used as placebo in this study.

### Administration

A single intravenous infusion of the diluted VB-111/placebo should be administered at 3 mL/minute. An infusion pump or syringe pump may be used.

The maximum time for VB-111/placebo in saline (until completion of administration) is 60 minutes (plus a 30-minute window) at room temperature.

Participants who weigh less than 50 kg will receive VB-111/placebo at a reduced dose (of 0.7×10¹³ VPs in 35 mL instead of 1×10¹³ VPs in 50 mL).

Expanded TCR clones, TIL Density and TCR Overlap

We will evaluate tumor and peripheral blood T cell receptor (TCR) repertoires with next generation sequencing in order to identify expanded and shared TCRs; thereby identifying anti-tumor immune responses induced by VB-111.

Genomic DNA will be isolated from fresh-frozen tumor (protocol surgery) and peripheral blood (immune monitoring time points) and analyzed with next generation sequencing through the TCRVβ region to quantify TIL density and assess the overlap between tumor and peripheral blood.

The number of peripherally expanded TCR clones, TIL density and TCR overlap will then be correlated with clinical variables to identify potential biomarkers with prognostic and predictive value for outcomes (PFS6, OS and toxicity).

### Immunohistochemistry (IHC) Measurements

A minimum of 1 formalin-fixed paraffin-embedded (FFPE) tumor tissue block (preferred) or a minimum of 10 FFPE unstained sections from pre-study surgery confirming GBM are to be submitted within 60 days of randomization. Additionally, a minimum 1 formalin-fixed paraffin-embedded (FFPE) tumor tissue block (preferred) or a minimum of 10 FFPE unstained sections from the protocol surgery are to be submitted per Table xx below. Multi-plex IHC stained will be performed to spatially quantitate PD-1, PD-L1, CD3, CD4, CD8, Iba-1, Ki-67 within the tumor microenvironment.

### Immunophenotyping

Tumor and peripheral blood T cell subsets and activation markers will be assessed with mass cytometry high dimensional analysis to identify immune cell subsets that expand or contract in an unbiased fashion during treatment with VB-111. The CD4/CD8 ratio, Treg populations (CD3+CD4+CD25+CD127low), activation (CD3+CD8+CD25/69), MDSC (CD33+HLA-DrlowCD11b+PD-L1+), negative costimulatory markers (CD3+CD4/8+PD-1+, CD3+CD4/8+CTLA-4+) will be determined at each time point. CyToF analysis will be performed on PBMC obtained from Ficoll density gradient separation of whole blood. Blood draws for this testing will be performed pre-treatment, pre-surgery, and with every MRI scan obtained for tumor status. Guidance on peripheral blood collection, processing and shipping is provided in Table 7 below.

### Cell-Cycle Related Gene Signatures, Gene Expression Signatures and Somatic Mutations

Tumor samples from the protocol surgery should be immersed in Allprotect tissue reagent solution (Qiagen). RNA-seq will be performed and analyzed. The number of somatic mutations in each tumor will be assessed, and data will be correlated with clinical variables to identify potential biomarkers with prognostic and predictive value for outcomes (PFS, OS). Cell-cycle related gene signatures will be assessed (Nanostring 10360) and sequencing data.

### Transgene Analysis

DNA and/or RNA will be extracted from the fresh frozen tissue sample using DNA and/or RNA isolation kits. DNA samples will be tested by PCR for the presence of the sequence of the inserted viral trans-gene in the tissue. RNA samples will be tested by PCR for viral trans-gene expression in the tissue.

TABLE 7
Correlative Sample Summary

| Biomarker | | Tissue/Body Fluid Tested (Timing of | Mandatory/ |
|---|---|---|---|
| name | Assay | Assay) | Optional |
| Expanded TCR | immunoSEQ Assay | Tumor (at the time of on study) and | M |
| clones | (Adaptive | peripheral blood (pre- Neo-adjuvant | |
| (integrated | Biotechnologies) | treatment, at the time of surgery, pre-dose | |
| biomarker) | on tumor tissue and | on D1 of post-surgery adjuvant treatment | |
| | PBMC genomic | cycle 1, and with every MRI during the | |
| | DNA | adjuvant treatment) | |
| TIL Density | immunoSEQ Assay | Tumor (at the time of on study surgery) and | M |
| and TCR | (Adaptive | peripheral blood (pre- Neo-adjuvant | |
| Clonality | Biotechnologies) | treatment, at the time of surgery, pre-dose | |
| | on tumor tissue and | on D1 of post-surgery adjuvant treatment | |
| | PBMC genomic | cycle 1, and with every MRI during the | |
| | DNA | adjuvant treatment) | |

TABLE 8
Exploratory Biomarkers Table

| Biomarker | | Tissue/Body Fluid Tested (Timing of | Mandatory/ |
|---|---|---|---|
| name | Assay | Assay) | Optional |
| Cell cycle-related | RNA-seq and | Tumor (protocolat the time of on study | O |
| gene signature | Nanostring IO360 | surgery) | |
| (integrated | | | |
| biomarker) | | | |
| IHC | PD-1/PD- | Tumor 10 unstained slides (archived | O |
| measurements | L1/CD3/CD4/CD8/Iba- | and protocol surgery) | |
| | 1/Ki-67 IHC on FFPE | | |
| | tumor tissue | | |
| Peripheral blood | Mass Cytometry on | Peripheral blood (pre- Neo-adjuvant | O |
| T cell | PBMC | treatment, at the time of surgery, pre- | |
| subsets/activation | | dose on D1 of post-surgery adjuvant | |
| markers | | treatment, cycle 1, and with every MRI | |
| | | during the adjuvant treatment) | |
| Gene expression | RNA Seq on tumor | Tumor (protocolat the time of on study | O |
| signatures and | RNA | surgery) and peripheral blood | |
| somatic | Exome sequencing on | (protocolat the time of on study | |
| mutations | normal/tumor gDNA | surgery) | |

TABLE 9
Exploratory Transgene Expression Table

| Biomarker | | Tissue/Body Fluid Tested (Timing | Mandatory/ |
|---|---|---|---|
| name | Assay | of Assay) | Optional |
| Cell cycle- | PCR and | Tumor (at the time of on study | O |
| related gene | qPCR | surgery) | |
| signature | | | |
| (integrated | | | |
| biomarker) | | | |

TABLE 10
Imaging Correlates Table

| Biomarker | | Tissue/Body Fluid Tested (Timing of | Mandatory/ |
|---|---|---|---|
| name | Assay | Assay) | Optional |
| Accurate | MRI | Brain (pre- Neo-adjuvant treatment, Pre- | M |
| distinction | (ADC, | surgery, pre-VB-111, post-surgery, post- | |
| between | Per- | VB-111 every 8 weeks during adjuvant | |
| tumor and | fusion) | treatment, EOT) | |
| inflamma- | | | |
| tion | | | |

### Measurement of Effect

Tumor response will be assessed every 8 weeks using contrast and non-contrast brain magnetic resonance imaging (MRI) with assessment based on the RANO criteria until progression is supported by two consecutive time points of tumor growth (local and central blinded independent radiology review). For participants who do not progress or die, PFS will be censored at the last adequate radiologic assessment. Baseline scan for determining progression will be the post-operative scan obtained just prior to initiating post-operative therapy.

### Measurable Disease

In some aspects, measurable disease is bi-dimensionally, contrast-enhancing, measurable lesions with clearly defined margins by MRI, with two perpendicular diameters of at least 10 mm, visible on 2 or more axial slices which are preferably at most 5 mm apart with 0 mm skip. The presence of inter-slice gaps should be considered in determining the size of measurable lesions at baseline. Measurement of tumor around a cyst or surgical cavity should be considered non-measurable unless there is a nodular component measuring at least 10 mm in diameter. The cystic or surgical cavity should not be measured in determining response. All tumor measurements must be recorded in millimeters.

### Non Measurable Disease

In some aspects, non-measurable disease is either uni-dimensionally measurable lesions, masses with margins not clearly defined, or lesions with maximal perpendicular diameter<10 mm.

### Target Lesions

In some aspects, all measurable lesions up to a maximum of 5 lesions should be identified as target lesions and recorded and measured (sum of the products of the perpendicular diameters) at baseline. Target lesions should be selected on the basis of their size (lesions with the longest diameters) and their suitability for accurate repeated measurements by imaging techniques. Occasionally, the largest lesions may not be suitable for reproducible measurement and the next largest lesions which can be measured reproducibly should be selected.

### Non-Target Lesions

For participants with recurrent disease who have multiple lesions of which only one or two are increasing in size, the enlarging lesions should be considered the target lesions for evaluation of response. The other lesions will be considered non-target lesions and should also be recorded.

Rarely, unequivocal progression of a non-target lesion requiring discontinuation of therapy, or development of a new contrast-enhancing lesion may occur even in the setting of stable disease (SD) or partial response (PR) in the target lesions. These changes would qualify as progression.

Non-target lesions also include measurable lesions that exceed the maximum number of 5. Measurements of these lesions are not required but the presence or absence of each should be noted throughout follow-up.

### Guidelines for Evaluation of Measurable Disease

All measurements should be taken and recorded in metric notation using a ruler or calipers. All baseline evaluations should be performed up to 28 days before the beginning of the treatment.

Measurement is performed by conventional MM. The same method of assessment and the same technique should be used to characterize each identified and reported lesion at baseline and during follow-up. These techniques should be performed with contiguous cuts of 10 mm or less in slice thickness. The Mills will be evaluated both locally and centrally by a core lab.

### Response Criteria for Target Lesions

### Complete Response (CR):

All of the following criteria must be met:
Complete disappearance of all enhancing measurable and non-measurable disease sustained for at least 4 weeks;
No new lesions;
Stable or improved non-enhancing (T2/FLAIR) lesions;
Participants must be off corticosteroids; and
Stable or improved clinically.

### Partial Response (PR):

All of the following criteria must be met:
Greater than or equal to 50% decrease compared to baseline in the sum of products of perpendicular diameters of all measurable enhancing lesions sustained for at least 4 weeks;
No progression of non-measurable disease;
No new lesions;
Stable or improved non-enhancing (T2/FLAIR) lesions on same or lower dose of corticosteroids compared to baseline scan;
The corticosteroid dose at the time of the scan evaluation should be no greater than the dose at time of the baseline scan; and
Stable or improved clinically.

### Stable Disease (SD):

All of the following criteria must be met:
Does not qualify for CR, PR, or progression;
Stable non-enhancing (T2/FLAIR) lesions on same or lower dose of corticosteroids compared to baseline scan; and
Stable clinically.

### Progressive Disease (PD):

Any of the following criterion must be met:
≥25% increase in the sum of products of perpendicular diameters of enhancing lesions compared to the smallest tumor measurement obtained either at baseline (if no decrease) or best response, on stable or increasing doses of corticosteroids;
Significant increase in T2/FLAIR non-enhancing lesion on stable or increasing doses of corticosteroids compared to baseline scan or best response following initiation of therapy, not due to co-morbid events (e.g. radiation therapy, demyelination, ischemic injury, infection, seizures, post-operative changes, or other treatment effects);
Any new lesion;
Clear clinical deterioration not attributable to other causes apart from the tumor (e.g. seizures, medication side effects, complications of therapy, cerebrovascular events, infection, etc.) or changes in corticosteroid dose;
Failure to return for evaluation due to death or deteriorating condition; or
Clear progression of non-measurable disease.

The RANO Response Criteria are summarized in Table 11:

TABLE 11
Summary of RANO Response Criteria

| | CR | PR | SD | PD# |
|---|---|---|---|---|
| T1-Gd+ | None | ≥50% decrease | <50% decrease to | ≥25% increase* |
| | | | <25% increase | |
| T2/FLAIR | Stable or decrease | Stable or decrease | Stable or decrease | Increase* |
| New Lesion | None | None | None | Present* |
| Corticosteroids | None | Stable or decrease | Stable or decrease | NA |
| Clinical Status | Stable or increase | Stable or increase | Stable or increase | Decrease* |
| Requirement for | All | All | All | Any* |
| Response | | | | |

CR = complete response;
PR = partial response;
SD = stable disease;
PD = progressive disease;
NA = not applicable
#Progression occurs when any of the criteria with * is present
NA Increase in corticosteroids alone will not be taken into account in determining progression in the absence of persistent clinical deterioration.

### Confirmatory Measurement/Duration of Response

Confirmation

To be assigned a status of PR or CR, changes in tumor measurements must be confirmed by repeat assessment that should be performed at least 4 weeks after the criteria for response are first met.

### Duration of Overall Response

The duration of overall response is measured from the time criteria are met for CR or PR until the first date that recurrent or progressive disease is objectively documented (taking as reference for progressive disease the smallest measurements recorded since the treatment started).

### Duration of Stable Disease

Stable disease is measured from the start of the treatment until the criteria for progression are met, taking as reference the smallest measurements recorded since the treatment started.

### Modification to RANO Criteria

The following adaptations of the RANO criteria will be used to assess response for participants treated on this study in an exploratory fashion (Table 12):

Potential Pseudoprogression: If radiologic imaging shows initial PD, participants who are not experiencing significant clinical decline (e.g. significant decrease in KPS), may be allowed to continue study treatment until progression is supported by two consecutive time points of tumor growth. Participants should be closely monitored with MRIs every cycle (approximately every 8 weeks) during this period. Participants who have radiographic evidence of further progression after up to three months, or who decline significantly at any time, will be classified as progressive with the date of disease progression back-dated to the first date that the participant met criteria for progression and such participants will be discontinued from study therapy. Three months is reasonable based on: 1) the peak time for XRT/daily temozolomide-related pseudoprogression is usually within three months of completion for glioblastoma participants and; 2) three months is also the most common timeframe for pseudoprogression observed among participants with advanced melanoma or other solid tumors treated with immune therapies such as immune checkpoint blockade to date.

Among participants on this study with initial radiographic PD, tumor assessment should be repeated regularly (every cycle, approximately every 8 weeks) in order to confirm PD with the option of continuing treatment and the addition of bevacizumab while awaiting radiologic confirmation of progression.

Participants who have progression that is supported by two consecutive time points of tumor growth will discontinue study medication and enter the follow up/survival phase of the study. In determining whether or not the tumor burden has increased or decreased, investigators should consider all target lesions as well as non-target lesions.

Tumor Enhancement to Define Progression: Radiographic progressive disease will be defined by assessment of enhancing tumor and will not declare tumor progression based on the presence of T2 or FLAIR changes alone as outlined in RANO.

In participants who have initial evidence of radiographic PD, the participant may continue on study treatment for up to three months pending confirmation of PD on follow-up imaging. Participants may receive bevacizumab 10 mg/kg every 2 weeks in addition to study treatment while waiting for confirmation of PD if they are not experiencing significant clinical decline and the participant is adequately tolerating study therapy (if a participant is required to discontinue study treatment for toxicity, then they must be taken off-treatment).

When feasible, study therapy should not be discontinued until progression is supported by two consecutive time points of tumor growth. This allowance to continue treatment despite initial radiologic progression takes into account the observation that some participants can have a transient tumor flare in the first few months after the start of immunotherapy, but with subsequent disease response. Participants that are exhibiting significant neurologic decline are not required to have repeat imaging for confirmation of progressive disease.

TABLE 12
Imaging and Treatment After First Radiologic Evidence of PD

| | No Significant Neurologic Decline | | Significant Neurologic Decline | |
|---|---|---|---|---|
| | Imaging | Treatment | Imaging | Treatment |
| 1^{st} radiologic | Repeat imaging | May continue study | Repeat imaging | May continue study |
| evidence of PD | (every cycle, | treatment at the | >6 weeks later | treatment and start |
| | approximately | Investigator's | to | bevacizumab |
| | every 8 weeks) | discretion for up to 3 | confirm PD if | 10 mg/kg Q2 weeks |
| | for up to 3 months | months while | possible | until the next |
| | to confirm PD | awaiting | | scheduled imaging |
| | | confirmatory | | assessment |
| | | imaging assessment | | |
| Repeat scan up | No additional | Discontinue | No additional | Discontinue treatment |
| to 3 months | imaging required; | treatment | imaging | |
| after 1^{st} | date of tumor | | required; date | |
| radiologic | progression back- | | of tumor | |
| evidence | dated to date of | | progression | |
| confirms PD | initial radiographic | | back-dated to | |
| | PD | | date of initial | |
| | | | radiographic | |
| | | | PD | |
| Repeat scan | Continue | Continue study | Continue | May continue study |
| shows SD, PR | regularly | treatment at the | regularly | treatment and |
| or CR | scheduled | Investigator's | scheduled | continue |
| | imaging | discretion | imaging | bevacizumab |
| | assessments | | assessments | indefinitely |
| | every 8 weeks | | every | |
| | | | 8 weeks | |

Participants with progressive radiographic findings are encouraged to undergo surgical intervention in order to delineate pseudoprogression due to inflammation associated with VB-111 from true tumor progression. Participants with histopathologic findings of significant immune infiltrate and evolving gliosis will be allowed to continue study therapy. In contrast, those with clear evidence of progressive tumor by histopathologic evaluation will be defined as progressive and discontinued from study therapy. For such participants, the date of tumor progression will be the first date the participant met radiographic criteria for PD.

### Radiology Review

The review of the baseline neuroimaging will be performed to ensure the participants meet the inclusion criterial before the randomization. The review of on-study neuroimaging will be performed retrospectively.

### Statistical Analysis Plan

This section outlines the statistical analysis strategy and procedures for the study. If, after the study has begun, changes are made to primary and/or key secondary hypotheses, or the statistical methods related to those hypotheses, then the protocol will be amended. Changes to exploratory or other non-confirmatory analyses made after the protocol has been finalized, along with an explanation as to when and why they occurred, will be listed in the Clinical Study Report (CSR) for the study. Post hoc exploratory analyses will be clearly identified in the CSR.

### Study Endpoints

Efficacy and safety endpoints that will be evaluated for within- and/or between-treatment differences are listed below, followed by the descriptions of the derivations of selected endpoints.

### Efficacy Endpoints

Tumor infiltrating T cell (TIL) density.

PFS6 is the percentage of participants with progression-free survival at 6 months as defined by RANO.

OS is the time from randomization until death from any cause. Participants will be followed for survival status after progression or discontinuation of the study drug for other reasons.

### Safety Endpoints

The primary safety endpoints are AEs graded using CTCAE version 5.0 criteria.

Safety will be assessed by quantifying the toxicities and grades experienced by participants who have received VB-111, including serious adverse events (SAEs) and events of clinical interest (ECIs). Other safety endpoints include laboratory safety assessments, KPS status, vital signs and physical examinations.

### Definitions of Analysis Sets

The Full Analysis Set population will serve as the population for the analysis of primary efficacy endpoint TIL density in this study. The Full Analysis Set population consists of all participants within each cohort who have received a preoperative dose of VB-111/placebo and had a surgery.

The intention to treat (ITT) population is defined as all randomized participants. The ITT population will be used for secondary efficacy endpoints (PFS6, OS).

The All Participants as Treated (APaT) population will be used for the analysis of safety data in this study. The APaT population consists of all allocated participants who received at least one dose of study treatment. At least one laboratory or vital sign measurement obtained subsequent to at least one dose of study treatment is required for inclusion in the analysis of each specific parameter. To assess change from baseline, a baseline measurement is also required.

### Sample Size Consideration

This is a randomized, controlled, blinded, phase II, surgical trial to evaluate early immunologic pharmacodynamic parameters for the viral cancer therapy VB-111 in participants with surgically accessible recurrent/progressive glioblastoma (rGBM). Participants will be randomly assigned to group A or B or C with a 1:1:1 randomization ratio prior to surgery. VB-111 can generate effective anti-tumor immune responses in the form of a statistically significant increase in TIL density comparing Group A (neoadjuvant/adjuvant) vs combined control group (Group B+C).

Based on our preliminary data, the mean of TIL density is estimated to be 0.4 (T cells per nucleated cell) (SD=0.5) in the combined control group (Group B and C). Comparing Group A vs Group B+C at an alpha level of 0.05 (1-sided) using a stratified two-sample t-test, a sample size of 30 in the combined Group (B+C), and 15 in Group A (Total of 45 evaluable) allows for the detection of a standard difference in TIL density of 0.8 or larger with 84% power. Effect size is defined as Cohen's D, the difference of two population means, divided by the pooled standard deviation.

### Efficacy Analysis

A two-sample t-test with tumor size stratification will be used to test the difference of tumor infiltrating T cell density between the two groups (Group A vs combined Group B+C). Data distribution will be examined prior to analysis. Data transformations will be performed as deemed appropriate.

For the secondary endpoint of PFS6, historical comparison data are available from a pooled analysis of Phase II experience in recurrent Grade IV gliomas who have undergone surgery either just prior to starting treatment or as part of the protocol, which documented a PFS6 rate of 10%. Group comparisons (Group A vs C; Group B vs C; Group A+B vs C) will be conducted using the exact binomial test with Bonferroni adjustment to compare PFS6 rate at one sided alpha of 5%.

For OS, analyses will be conducted using historical comparison data which are available from a pooled analysis of Phase II experience in recurrent Grade IV gliomas who have undergone surgery either just prior to starting treatment or as part of. Kaplan-Meier (KM) curves and median estimates from the KM curves will be provided as appropriate. Participants without efficacy evaluation data or without survival data will be censored at Day 1. For evaluation of the expansion of TCR clones, a two-sample T-test with Bonferroni adjustment will be used to compare the increase number of expanded TCR clones after VB-111 in Group A+B vs Group C.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present disclosure. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

What is claimed:

1. A vector comprising a Fas-chimera gene operably linked to an endothelial cell specific promoter for use in inducing or improving an anti-tumor response in a subject having a tumor, wherein the subject is to be administered a priming dose of the vector prior to surgical removal of the tumor or a portion thereof, and wherein the anti-tumor response is induced or improved after the administration compared to the anti-tumor response in a subject not receiving a priming dose of the vector prior to surgical removal of the tumor or a portion thereof or in a subject not administered the vector.

2. A vector comprising a Fas-chimera gene operably linked to an endothelial cell specific promoter for use in treating a tumor in a subject in need thereof, wherein the subject is to be administered a priming dose of the vector prior to surgical removal of the tumor or a portion thereof.

3. The vector for use of claim 1 or 2, wherein the subject is to be further administered a post-surgical dose of the vector.

4. The vector for use of claim 3, wherein the post-surgical dose of the vector is to be further administered in combination with one or more chemotherapeutic agents.

5. The vector for use of any one of claims 1 to 4, wherein the priming dose of the vector is to be administered about 7 days, about 8 days, about 9 days, about 10 days, about 11 days, about 12 days, about 13 days, about 2 weeks, about 15 days, about 20 days, about 3 weeks, about 25 days, about 4 weeks, about a month, about 5 weeks, about 6 weeks, about 7 weeks, about 2 months, about 3 months, about 4 months, about 5 months, or about 6 months before surgical removal of the tumor or a portion thereof.

6. The vector for use of any one of claims 1 to 5, wherein the priming dose of the vector is to be administered at an effective amount of less than about 1×10¹⁵, less than about 1×10¹⁴, less than about 5×10¹³, less than about 4×10¹³, less than about 3×10¹³, less than about 2×10¹³, less than about 1×10¹³, less than about 9×10¹², less than about 8×10¹², less than about 7×10¹², less than about 6×10¹², less than about 5×10¹², less than about 4×10¹², less than about 3×10¹², less than about 2×10¹², less than about 1×10¹², less than about 9×10¹¹, less than about 8×10¹¹, less than about 7×10¹¹, less than about 6×10¹¹, less than about 5×10¹¹, less than about 4×10¹¹, less than about 3×10¹¹, less than about 2×10¹¹, less than about 1×10¹¹, less than about 9×10¹⁰, less than about 8×10¹⁰, less than about 7×10¹⁰, less than about 6×10¹⁰, less than about 5×10¹⁰, less than about 4×10¹⁰, less than about 3×10¹⁰, less than about 2×10¹⁰, or less than about 1×10¹⁰ virus particles.

7. The vector for use of any one of claims 3 to 5, wherein the post-surgical dose of the vector is to be administered at an effective amount of less than about 1×10¹⁵, less than about 1×10¹⁴, less than about 5×10¹³, less than about 4×10¹³, less than about 3×10¹³, less than about 2×10¹³, less than about 1×10¹³, less than about 9×10¹², less than about 8×10¹², less than about 7×10¹², less than about 6×10¹², less than about 5×10¹², less than about 4×10¹², less than about 3×10¹², less than about 2×10¹², less than about 1×10¹², less than about 9×10¹¹, less than about 8×10¹¹, less than about 7×10¹¹, less than about 6×10¹¹, less than about 5×10¹¹, less than about 4×10¹¹, less than about 3×10¹¹, less than about 2×10¹¹, less than about 1×10¹¹, less than about 9×10¹⁰, less than about 8×10¹⁰, less than about 7×10¹⁰, less than about 6×10¹⁰, less than about 5×10¹⁰, less than about 4×10¹⁰, less than about 3×10¹⁰, less than about 2×10¹⁰, or less than about 1×10¹⁰ virus particles.

8. The vector for use of any one of claims 3 to 7, wherein the priming dose of the vector and the post-surgical dose of the vector are the same.

9. The vector for use of any one of claims 3 to 7, wherein the priming dose of the vector and the post-surgical dose of the vector are different.

10. The vector for use of any one of claims 3 to 9, wherein the post-surgical dose of the vector is to be repeatedly administered every day, once in about 2 days, once in about 3 days, once in about 4 days, once in about 5 days, once in about 6 days, once in about 7 days, once in about 2 weeks, once in about 3 weeks, once in about 4 weeks, once in about 5 weeks, once in about 6 weeks, once in about 7 weeks, once in about 2 months, or once in about 6 months.

11. The vector for use of any one of claims 1 to 10, wherein the tumor is derived from or associated with a sarcoma, melanoma, carcinoma, leukemia, Hodgkin's Disease, Non-Hodgkin's Lymphoma, multiple myeloma, neuroblastoma, breast cancer, ovarian cancer, lung cancer (including non-small cell lung cancer (NSCLC)), rhabdomyosarcoma, primary thrombocytosis, primary macroglobulinemia, small-cell lung tumors, primary brain tumors, gliomas (including glioblastoma multiforme (GBM) and recurrent GBM), stomach cancer, colon cancer (including metastatic colorectal cancer (mCRC)), hepatobiliary cancer, malignant pancreatic insulanoma, malignant carcinoid, urinary bladder cancer, premalignant skin lesions, testicular cancer, lymphomas, thyroid cancer, papillary thyroid cancer, neuroblastoma, neuroendocrine cancer, esophageal cancer, genitourinary tract cancer, malignant hypercalcemia, cervical cancer, endometrial cancer, adrenal cortical cancer, prostate cancer, Müllerian cancer, peritoneal cancer, fallopian tube cancer, or uterine papillary serous carcinoma.

12. The vector for use of claim 11, wherein the tumor is a recurrent glioblastoma.

13. The vector for use of any one of claims 1 to 12, wherein the vector comprises, consists of, or consists essentially of SEQ ID NO: 19.

14. The vector for use of any one of claims 1 to 13, wherein the vector is an isolated virus having European Collection of Cell Cultures (ECACC) Accession Number 13021201.

15. The vector for use of any one of claims 4 to 14, wherein the one or more chemotherapeutic agents is bevacizumab.
